SEQUENCE LISTING (21 sequences)

SEQ ID NO: 1 - DNA, 21 bp, Artificial
  Primer
atggggacaaaggcgcaagtc

SEQ ID NO: 2 - DNA, 19 bp, Artificial
  Primer
caccaggaatgacgaggtc

SEQ ID NO: 3 - DNA, 25 bp, Artificial
  Primer
atacaagtgtatgtcccagtgtctt

SEQ ID NO: 4 - DNA, 31 bp, Artificial
  Primer
attgcggccgcgatgaaatgccccagtgccg

SEQ ID NO: 5 - DNA, 31 bp, Artificial
  Primer
ccaagcttgctattggtcggatggatcctcg

SEQ ID NO: 6 - DNA, 56 bp, Artificial
  Primer
gcagggtggtctgatcctcagtgatggtgatggtgatgcgtgaaactacgcgggta

SEQ ID NO: 7 - DNA, 39 bp, Artificial
  Primer
gatgaaatgccccagtgccgcggggtggtctgatcctca

SEQ ID NO: 8 - DNA, 18 bp, Artificial
  Primer
gatgaaatgccccagtgc

SEQ ID NO: 9 - DNA, 20 bp, Artificial
  Primer
gagcatgtggataggaattg

SEQ ID NO: 10 - DNA, 18 bp, Artificial
  Primer
gtgatggtgatggtgatg

SEQ ID NO: 11 - DNA, 16 bp, Artificial
  Primer
gtaaaacgacggccag

SEQ ID NO: 12 - DNA, 17 bp, Artificial
  Primer
caggaaacagctatgac

SEQ ID NO: 13 - DNA, 1416 bp, Reovirus sp.
gctattggtcggatggatcctcgcctacgtgaagaagtagtacggctgataatcgcattaacgagtgataatggagtatcactgtcaaaagggcttgaatcaagggtctcggcgctcgagaagacgtctcaaatacactctgatactatcctccggatcacccagggactcgatgatgcaaacaaacgaatcatcgctcttgagcaaagtcgggatgacttggttgcatcagtcagtgatgctcaacttgcaatctccagattggaaagctctatcggagccctccaaacagttgtcaatggacttgattcgagtgttacccagttgggtgctcgagtgggacaacttgagacaggacttgcagagctacgcgttgatcacgacaatctcgttgcgagagtggatactgcagaacgtaacattggatcattgaccactgagctatcaactctgacgttacgagtaacatccatacaagcggatttcgaatctaggatatccacattagagcgcacggcggtcactagcgcgggagctcccctctcaatccgtaataaccgtatgaccatgggattaaatgatggactcacgttgtcagggaataatctcgccatccgattgccaggaaatacgggtctgaatattcaaaatggtggacttcagtttcgatttaatactgatcaattccagatagttaataataacttgactctcaagacgactgtgtttgattctatcaactcaaggacaggcgcaattgagcaaagttacgtggcgtcggcagtgactcccttgagattaaacagtagcacgaaggtgctggatatgctaatagacagttcaacacttgaaattaattctagtggacagctaactgttagatcgacatccccgaatttgaggtatccgatagctgatgttagcggcggtatcggaatgagtccaaattataggtttaggcagagcatgtggataggaattgtctcctattctggtagtgggctgaattggagggtacaggtgaactccgacatttttattgtagatgattacatacatatatgtcttccagcttttgacggtttctctatagctgacggtggagatctatcgttgaactttgttaccggattgttaccaccgttacttacaggagacactgagcccgcttttcataatgacgtggtcacatatggagcacagactgtagctatagggttgtcgtcgggtggtgcgcctcagtatatgagtaagaatctgtgggtggagcagtggcaggatggagtacttcggttacgtgttgaggggggtggctcaattacgcactcaaacagtaagtggcctgccatgaccgtttcgtacccgcgtagtttcacgtgaggatcagaccaccccgcggcactggggcatttcatc

SEQ ID NO: 14 - protein, 455 aa, Reovirus sp.
MDPRLREEVVRLIIALTSDNGVSLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQIVNNNLTLKTTVFDSINSRTGAIEQSYVASAVTPLRLNSSTKVLDMLIDSSTLEINSSGQLTVRSTSPNLRYPIADVSGGIGMSPNYRFRQSMWIGIVSYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFT

SEQ ID NO: 15 - DNA, 1434 bp, Reovirus sp.
gctattggtcggatggatcctcgcctacgtgaagaagtagtacggctgataatcgcattaacgagtgataatggagtatcactgtcaaaagggcttgaatcaagggtctcggcgctcgagaagacgtctcaaatacactctgatactatcctccggatcacccagggactcgatgatgcaaacaaacgaatcatcgctcttgagcaaagtcgggatgacttggttgcatcagtcagtgatgctcaacttgcaatctccagattggaaagctctatcggagccctccaaacagttgtcaatggacttgattcgagtgttacccagttgggtgctcgagtgggacaacttgagacaggacttgcagagctacgcgttgatcacgacaatctcgttgcgagagtggatactgcagaacgtaacattggatcattgaccactgagctatcaactctgacgttacgagtaacatccatacaagcggatttcgaatctaggatatccacattagagcgcacggcggtcactagcgcgggagctcccctctcaatccgtaataaccgtatgaccatgggattaaatgatggactcacgttgtcagggaataatctcgccatccgattgccaggaaatacgggtctgaatattcaaaatggtggacttcagtttcgatttaatactgatcaattccagatagttaataataacttgactctcaagacgactgtgtttgattctatcaactcaaggacaggcgcaattgagcaaagttacgtggcgtcggcagtgactcccttgagattaaacagtagcacgaaggtgctggatatgctaatagacagttcaacacttgaaattaattctagtggacagctaactgttagatcgacatccccgaatttgaggtatccgatagctgatgttagcggcggtatcggaatgagtccaaattataggtttaggcagagcatgtggataggaattgtctcctattctggtagtgggctgaattggagggtacaggtgaactccgacatttttattgtagatgattacatacatatatgtcttccagcttttgacggtttctctatagctgacggtggagatctatcgttgaactttgttaccggattgttaccaccgttacttacaggagacactgagcccgcttttcataatgacgtggtcacatatggagcacagactgtagctatagggttgtcgtcgggtggtgcgcctcagtatatgagtaagaatctgtgggtggagcagtggcaggatggagtacttcggttacgtgttgaggggggtggctcaattacgcactcaaacagtaagtggcctgccatgaccgtttcgtacccgcgtagtttcacgcatcaccatcaccatcactgaggatcagaccaccccgcggcactggggcatttcatc

SEQ ID NO: 16 - protein, 461 aa, Reovirus sp.
MDPRLREEVVRLIIALTSDNGVSLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQIVNNNLTLKTTVFDSINSRTGAIEQSYVASAVTPLRLNSSTKVLDMLIDSSTLEINSSGQLTVRSTSPNLRYPIADVSGGIGMSPNYRFRQSMWIGIVSYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFTHHHHHH

SEQ ID NO: 17 - protein, 461 aa, Reovirus sp.; identical to SEQ ID NO: 16

SEQ ID NO: 18 - protein, 461 aa, Reovirus sp.; identical to SEQ ID NO: 16

SEQ ID NO: 19 - protein, 461 aa, Reovirus sp.; identical to SEQ ID NO: 16

SEQ ID NO: 20 - protein, 461 aa, Reovirus sp.
MDPRLREEVVRLIIALTSDNGVSLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQIVNNNLTLKTTVFDSINSRTGAIEQSYVASAVTPLRLNSSTKVLDMLIDSSTLEINSSGQLTVRSTSPNLRYPIADVSGGIGMSPNYRFRQSMWIGIVYYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFTHHHHHH

SEQ ID NO: 21 - protein, 461 aa, Reovirus sp.
MDPRLREEVVRLIIALTSDNGVSLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNGGLTLSGNNLAIRLPGNTGLNIQNGGLQFRFNTDQFQIVNNNLTLKTTVFDSINSRTGAIEQSYVASAVTPLRLNSSTKVLDMLIDSSTLEINSSGQLTVRSTSPNLRYPIADVSGGIGMSPNYRFRQSMWIGIVYYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFTHHHHHH

(19) United States
(12) Patent Application Publication
(10) Pub. No.: US 2010/0278863 A1
(43) Pub. Date: Nov. 4, 2010

(54) Reoviruses

(75) Inventors: Robert Cornelis Hoeben, Leiden (NL); Diana Johanna Maria Van Den Wollenberg, Noordwijk (NL)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Academisch Zeikenhuis Leiden Leids Universitair Medisch Centrum, Leiden (NL)

(21) Appl. No.: 12/738,814

(22) PCT Filed: Oct. 16, 2008
(86) PCT No.: PCT/IB2008/002758

§ 371 Date: Jul. 23, 2010

(30) Foreign Application Priority Data
Oct. 20, 2007 (GB) 07020624.6

(51) Int. Cl. A61K 39/15; C12N 15/00; C12N 7/01; A61K 35/76; A61P 35/00; A61P 37/00

(52) U.S. Cl. 424/215.1; 435/440; 435/235.1; 424/93.2

(57) ABSTRACT

The present invention provides a reverse genetics system for viruses belonging to the Reoviridae (i.e. Reoviruses), various uses thereof, genetically modified Reoviruses, Reovirus selection/production and propagation systems, medicaments and vaccines.

### FIELD OF THE INVENTION

The present invention provides a reverse genetics system for viruses belonging to the Reoviridae (i.e. Reoviruses), various uses thereof, genetically modified Reoviruses, Reovirus selection/production and propagation systems, medicaments and vaccines.

### BACKGROUND

The Reoviridae (Respiratory Enteritic Orphan viruses) constitute a family of non-enveloped viruses with segmented double-stranded RNA genomes. The Reoviridae family includes viruses that affect the gastrointestinal system (such as the Rotaviruses), which cause respiratory infections. The term “orphan virus” indicates that a particular virus is not associated with any known disease and, while Reoviridae have been associated with a number of diseases, the original name is still used (Tyler, 2001). Rotaviruses can be transmitted directly from human to human and are the major etiologic agents of serious diarrhoeal illness in children under 2 years of age throughout the world, resulting in approx. 500,000 deaths per annum (Kapikian et al., 2001).

Prototypes of the mammalian Orthoreoviruses have been isolated from the human respiratory and enteric tracts, but are not associated with serious human disease. One of these, human Reovirus type 3 Dearing (T3D), is studied frequently and usually serves as a model for the family (Nibert et al., 2001). Moreover, during the last decade the mammalian Orthoreoviruses, especially T3D, have been used as an oncolytic agent in preclinical and clinical cancer therapy experiments (Norman et al., 2000; Shmulevitz et al., 2005). The present invention is based, in part, on the observation that Reoviruses induce cell death and apoptosis in tumor cells, but not in healthy non-transformed cells (Hashiro et al., 1977; Duncan et al., 1978). To-date, several clinical trials have been initiated in Canada, the United States, and the United Kingdom, to study the feasibility of such an approach to cancer treatment.

Wild-type Reoviruses can use several distinct proteins as receptors for binding to its target cells. Firstly, the Junction Adhesion Molecule-A (Jam-A, also known as Junction Adhesion Molecule 1, or Jam-1) has been demonstrated to serve as the receptor for Orthoreoviruses type 1 and 3 and can mediate virus attachment and infection (Chappell et al., 2002d). Jam-A is an integral tight junction protein and a region in the globular head of the Sigma-1 protein of Reovirus T3D interacts with Jam-A (Chappell et al., 2002c). In addition, sequences in the shaft domain of the spike protein Sigma-1 can interact with cell surface sialic acid molecules for productive infection (Chappell et al., 1997). The Sigma-1 protein is encoded by the RNA segment S1 (also known as σ1).

Despite the common occurrence of Reovirus receptors, some tumor cells may have a limited number of receptors on their cell surface. For instance, Smakman (2005) described that none of the 13 tumor fragments from patients with colorectal metastases were susceptible to Reovirus T3D infection (Smakman, 2005). The scarcity of Reovirus receptors on tumor cells thwarts the efficiency of Reoviruses as oncolytic agents.

Genetic modification of Reoviridae is notoriously difficult due to the segmented structure of their double-stranded RNA genomes. As such, the present invention pertains to a reverse genetics method for members of the Reoviridae. Roner et al., 2001 & Roner et al., 1990 developed a complicated Reovirus reverse genetics system involving in-vitro synthesis of one of the RNA segments, in-vitro capping of this RNA, and co-transfection of this RNA with single-stranded (plus-stranded) and/or double-stranded RNA's of the other nine segments. To initiate replication the transfected cells were infected with the slow-plaqueing reovirus variant reovirus T2 or T1 as helper virus. (Roner et al., 2001) Although a single recombinant reovirus T3D which harbours a chloramphenicol-acetyltransferase gene was generated, the method is inefficient and cumbersome.

An alternative approach has been described by Komoto and Sasaki (Komoto et al., 2006), who describe the establishment of a reverse genetics system for rotaviruses. While they succeeded in rescuing re-assorted rotavirus, the method is very inefficient and needs to be improved. Kobayashi and collaborators recently described the generation of recombinant Reovirus T3D using a fully plasmid-base system. (Kobayashi, T., A. A. R. Antar, K. W. Boehme, P. Danthi, E. A. Eby, K. M. Guglielmi, G. H. Holm, E. M. Johnson, M. S. Maginnis, S. Naik, W. B. Skelton, J. D. Wetzel, G. J. Wilson, J. D. Chappell, and T. S. Dermody. 2007. A Plasmid-Based Reverse Genetics System for Animal Double-Stranded RNA Viruses. Cell Host & Microbe 1:147-157).

Both these methods rely on production of single-stranded plus-strand RNA with genuine segment termini. This thwarts the formation of recombinants since it is well known that in general non-polyadenylated single-stranded RNAs are very short-lived in mammalian cells. (Zeevi et al., 1982)

Accordingly, it is among the objects of the present invention to obviate or mitigate the abovementioned problems with the prior art.

### SUMMARY OF THE INVENTION

The present invention is based upon the development of an efficient reverse genetics system for Reoviridae which may have particular application in the development of, for example, genetically modified and/or host-range variants of Reoviruses.

In a first aspect, the present invention provides a method for modifying the genome of a virus belonging to the Reoviridae, said method comprising the steps of:

(a) introducing a nucleic acid encoding a modified portion of a Reovirus genome into a cell;

(b) infecting the cell with a Reovirus; and

(c) maintaining the cell under conditions which induce the production of modified virus;

wherein said modified virus comprises, relative to the Reovirus used in step (b), a modified genome comprising the modified portion of the Reovirus genome.

The present invention is based upon the surprising observation that, when expressed in a cell, modified portions of a Reovirus genome may be incorporated into newly formed Reovirus particles. Without wishing to be bound by theory, it is believed that the modified Reovirus genome portion, once introduced into the cell, is transcribed to yield a mRNA molecule which initiates at the genuine 5′ cap, is not truncated at the 3′ end and which is extended to further comprise a poly A tract.

The Reoviridae are a family of non-enveloped viruses (otherwise known as Reoviruses) having segmented double-stranded RNA genomes which includes, for example, Orthoreovirus, Orbivirus, Rotavirus and Coltivirus species. As such, the present invention provides a method of modifying the genomes of those viruses which belong to the Reoviridae family. In one embodiment, the present invention provides a method of genetically modifying the genome of Orthoreovirus species such as, for example, Reovirus type 3, strain Dearing (T3D).

Typically, the step of infecting a cell with a Reovirus (step (b) above) may require the use of a “wild-type Reovirus”. A wild-type Reovirus may be a native or naturally occurring form of any virus which belongs to the Reoviridae. Preferably, the wild-type Reovirus is a wild-type form of the Reovirus to be subjected to the methods described herein. By way of example, if the method concerns modifying the genome of an Orthoreovirus species, the Reovirus virus used to infect the cell may be a wild-type form of said Orthoreovirus species.

Alternatively, the step of infecting a cell with a Reovirus may be performed with Reovirus mutants or variants. For example, in certain embodiments it may be desirable to use, for example, heat sensitive and/or host-range mutants. Additionally, or alternatively, the Reovirus mentioned in step (b) above may be a Reovirus which, compared to a reference or wild-type strain of the same species, comprises a modified genome. In such cases the genome of the Reovirus used in step (b) of the methods described herein may be modified in accordance with any of the methods described herein or already known to one of skill in the art. As above, the variant, mutant or modified Reovirus used in step (b) is preferably a variant, mutant or modified version of the Reovirus to be subjected to the methods described herein. By way of example, if the method concerns modifying the genome of an Orbivirus species, the reovirus used to infect the cell may be a variant, mutant and/or modified form of said Orbivirus species.

It is to be understood that viruses subjected to the methods described herein are “modified” relative to the Reovirus used to infect the cell and that the Reovirus used in step (b) may be a wild-type, variant, mutant or modified form of the same virus. Accordingly, the phrase “modified genome” is intended to mean a genome which, when compared to the genome derived from the virus used in step (b), is altered or differs in some way. For example, a genome may be modified to contain additional nucleotides and/or substituted and/or inverted nucleotides. Additionally, or alternatively, the genome may be modified such that, relative to the genome of virus used to infect the cell, certain nucleotides are deleted.

Furthermore, the term “cell” encompasses any type of cell capable of being infected by a wild-type Reovirus. Well known examples include cell lines ‘911’, PER.C6, ‘293’, HeLa, A549, and L929.

In one embodiment, the methods described herein may be used to modify one or more of the double-stranded RNA genome segments which comprise the Reovirus genome. Additionally, or alternatively, the methods may be used to modify a portion or portions of one or more of the double-stranded RNA genome segments.

It is to be understood that the genome modification(s) introduced by the methods described herein, may manifest as one or more modification(s) in component(s) (for example one or more structural and/or non-structural proteins) of the virus produced by the cell infected in step (b) of the method according to the first aspect. In other words, the modified genome produced by the methods described herein may encode one or more modified viral component(s). Accordingly, in addition to providing a method capable of producing Reovirus having a modified genome, the present invention also provides a method of modifying one or more of the viral components encoded by the genome. In this way, virus produced by the cell infected in step (b) above, may comprise one or more modified component(s) (for example a structural and/or a non-structural protein) and/or a modified genome.

In a further embodiment, the method may be used to modify one or more the structural components such as those comprising, for example, the core or capsid structures. Additionally, or alternatively, the method may be used to modify one/or more of the non-structural components such as, for example, proteins involved in infection, replication, assembly and/or release. In particular, the method may be used to modify one or more of the proteins comprising the viral capsid.

Additionally, or alternatively, the methods described herein may be used to modify the Reovirus genome such that it comprises one or more heterologous nucleic acid sequence(s). In one embodiment, a heterologous nucleic acid sequence may encode a heterologous component and/or protein. For example, the genome may be modified to replace one or more of the native or natural Reovirus components with a corresponding heterologous component. In a further embodiment, the Reovirus genome may be modified such that it encodes one or more heterologous component(s) and/or protein(s) in addition to the native or natural components encoded for by the Reovirus genome.

In a yet further embodiment, the heterologous nucleic acid sequence may encode a compound or compounds which induce cell death or apoptosis or which may inhibit or suppress one or more cellular processes.

It is to be understood that the term “heterologous” refers to nucleic acid sequences and/or products thereof (for example proteins encoded thereby), derived from sources other than the particular Reovirus being subjected to the methods described herein.

In the case of the Orthoreovirus-Reovirus type 3, strain Dearing (T3D), in addition to modifying one or more of the double-stranded RNA segments comprising the genome (or a portion or portions thereof), any of the methods described herein may also be used to modify one or more of the components, for example the structural and/or non-structural components, of T3D. In particular, the methods may be used to modify one or more of the proteins comprising the T3D inner and/or outer capsid. Proteins Sigma1, Sigma3, Lambda2 and Mu1c are components of the outer capsid, and proteins Lambda1, Lambda3, Sigma2 and Mu2 are part of the inner capsid.

One of skill in the art will appreciate that, in order to modify a number of viral components, a number of nucleic acids—each encoding a modified component of the virus could be introduced into the cell.

Preferably, the component or components is/are are structural and/or non-structural component(s). For example, the structural component may be a protein comprising the viral capsid.

Preferably, and in one embodiment, the nucleic acid to be introduced into the cell may be provided by methods which comprise the step of generating a complementary DNA (cDNA) copy of a selected portion or selected portions of the genome of the virus. Advantageously, the selected portion or portion(s) of the viral genome may encode one or more components of the virus.

In one embodiment, a cell (a host cell) may be used to propagate the virus that is to be subjected to the methods described herein. Cells suitable for use as host cells may include for example, 911 cells, PER.C6 cells, 293 cells, HeLa cells, A549 cells, and L929 cells.

Typically, the cell in which the virus has been propagated may be subjected to a RNA extraction protocol. In one embodiment, the RNA extraction protocol may involve the step of subjecting the host cell to conditions which induce lysis. Such conditions may include the use of freeze-thawing the virus-containing cell suspension. In this way, any viral particles within the host cell may be released and harvested by, for example, centrifugation, preferably ultra-centrifugation.

Advantageously, the harvested virus particles may be subjected to conditions which induce lysis. Such conditions may include the use of, for example, chaotropic compounds capable of denaturing virus particles and inactivating enzymes which may otherwise denature and/or destroy nucleic acid. Such compounds may include, for example, urea and/or guanidinium compounds such as guanidinium chloride or guanidinium thiocyanate. Typically, residual viral and/or cellular debris may be removed by further rounds of centrifugation to leave a supernatant comprising viral RNA.

Preferably, RNA extraction may be achieved by way of nucleic acid precipitation techniques involving the use of compounds such as phenol-chloroform, silica beads, particles or diatoms and/or micro-spin columns designed to extract RNA from solutions (QIAGEN). Further information concerning these techniques may be obtained from, for example, Boom et al., Rapid and simple method for purification of nucleic acids, Journal of Clinical Microbiology, vol. (3)28, p 495-503; Shafer et al., Interlaboratory comparison of sequence-specific PCR and ligase detection reaction to detect a human immunodeficiency virus type 1 drug resistance mutation. The AIDS Clinical Trials Group Virology Committee Drug Resistance Working Group J. Clin. Microbiol. 1996 34: 1849-1853 and Molecular Cloning: A Laboratory Manual (Third Edition); Sambrook et al.; CSHL Press.

Preferably, the extracted RNA may be subjected to an amplification protocol in which oligonucleotide primers specific for a particular viral RNA sequence or sequences (referred to hereinafter as target viral sequence(s) are used to amplify a selected sequence or sequences. Typically, the oligonucleotide primers are designed to specifically hybridise with certain nucleotide sequences.

In one embodiment, the target viral sequence(s) encode certain viral structural components and/or non-structural components. For example, the target viral sequence(s) may encode one or more capsid proteins.

Advantageously, the oligonucleotide primers are contacted with the viral RNA under conditions which permit the generation of a cDNA copy of the target viral sequences. Such conditions may involve the use of enzymes capable of reverse transcribing RNA into cDNA. In one embodiment, the target sequence or sequences are amplified by reverse transcriptase polymerase chain reaction (RT-PCR). Further information concerning RT-PCR can be found in, for example, Molecular Cloning: A Laboratory Manual (Third Edition); Sambrook et al.; CSHL Press.

Preferably, and in one embodiment, the target viral sequence may be modified so as to provide a sequence which, when compared to the corresponding wild-type viral sequence, is altered or differs in some way. For example, the target viral sequence may be modified so as to comprise nucleotides which encode an amino acid sequence which, when compared to the corresponding amino acid sequence in a wild-type form of the virus, comprises one or more added, deleted, substituted or inverted amino acid residues.

Advantageously, the target viral sequence may be modified during the amplification protocol. Preferably, in addition to those nucleotides which specifically hybridise to the target sequence, the oligonucleotide primers for use in the RT-PCR amplification protocol described above, may further comprise a nucleotide sequence which encodes a modification to be introduced into the resultant cDNA. Additionally or alternatively, the oligonucleotide may comprise a nucleotide sequence that results in the deletion, substitution or inversion of one or more amino acids encoded by the viral target sequence.

Accordingly, the methods described herein may comprise the step of introducing into a cell a complementary DNA (cDNA) encoding a modified portion of a Reovirus genome and/or a modified component of a Reovirus.

The steps involved in introducing a nucleic acid into a cell are well known to one of skill in the art and may involve, for example, the use of transfection protocols or vectors (for example eukaryotic gene expression vectors) such as transcription cassettes, plasmids or viral vectors. Desirably the vector is not a vaccinia virus, T7 RND polymerase driven vector advantageously the present methods do not rely on the use of helper viruses.

Typically, transfection protocols utilise conditions which render cell membranes permeable to compounds such as nucleic acids. By way of example, it may be possible to transfect nucleic acid into cells using electroporation, heat shock and/or compounds such as calcium phosphate.

Additionally, or alternatively, the nucleic acid may be introduced into the cell by means of a gene gun. In such cases, the nucleic acid to be introduced may be associated with or otherwise conjugated to a particle which can be delivered directly to the cell.

Preferably, the nucleic acid to be introduced into the cell is contained within a RNA polymerase II-dependent transcription cassette such as, for example, a viral vector. In this way, the nucleic acid may be stably expressed. In one embodiment, the transcription cassette is capable of stably integrating into the genome of the cell such that the product of the introduced nucleic acid is stably expressed. Preferably, the RNA polymerase II-dependent transcription cassette is a lentiviral vector.

Thus, in one embodiment, the present invention provide a method for modifying the genome and/or a component of a virus belonging to the Reoviridae family, in which the nucleic acid (for example cDNA) is contained within a RNA polymerase II-dependent transcription cassette, such as for example, a vector.

Advantageously, the vector is a viral vector, preferably a lentiviral vector.

Virus belonging to the Reoviridae may bind to particular types of receptor molecule present on the surface of certain cells. For example, Junction Adhesion Molecule-A (JAM-A: otherwise known as Junction Adhesion Molecule 1, or Jam-1) is known to act as a receptor (mediating attachment and infection) for Orthoreoviruses type 1 and type 3. More specifically, a portion (a region of the globular head) of the T3D capsid protein Sigma-1 (S1) interacts with Jam-A while certain other sequences within the shaft domain of S1 may interact with sialic acid molecules present on the cell surface. Upon binding a particular cellular molecule (referred to hereinafter as a “cellular receptor”) the virus may be internalised and hence “infect” the cell.

Specific interactions between viral structural components and cellular receptors contribute to the particular cellular tropism (i.e. binding and infectivity specificity) exhibited by viruses belonging to the Reoviridae.

Accordingly, and in a further aspect, there is provided a method of modifying the cellular tropism of a virus belonging to the Reoviridae, said method comprising the steps of:

(a) introducing a nucleic acid encoding a modified component of a Reovirus into a cell;

(b) infecting the cell with a Reovirus; and

(c) maintaining the cell under conditions which induce the production of modified Reovirus of modified cellular tropism;

wherein said modified Reovirus of modified tropism comprises, relative to the Reovirus used in step (b), the modified component the Reovirus.

Preferably, the modified component of a Reovirus may be a modified structural component such as a viral capsid protein. Advantageously, the modification renders the viral component capable of binding a cellular receptor, which the Reovirus used in step (b) is unable to bind.

In a further embodiment, the method of modifying the cellular tropism of a virus belonging to the Reoviridae, may comprise the steps of modifying the genome of the virus such that it encodes a protein capable of binding to a particular cell. In this way it may be possible to target Reovirus particles to cells such as dendritic cells, macrophages and/or other types of immunological or white blood cell and/or cells derived from tissues and organs of the human or animal body.

In one embodiment, the present invention provides a method of modifying the cellular tropism of T3D, said method comprising the step of:

(a) providing a nucleic acid encoding a modified S1 capsid protein;

(b) introducing the nucleic acid into a cell;

(c) infecting the cell with a T3D virus; and

(d) maintaining the cell under conditions suitable which the production of new T3D virus of modified cellular tropism;

wherein said new T3D of modified tropism comprises, relative to the T3D virus used in step (c), said modified S1 capsid protein.

Typically, the T3D virus used in step (c) is a wild-type, mutant, variant or modified form of the T3D subjected to the above described method.

Preferably, the modified S1 protein comprises a modified primary structure which renders the S1 protein capable of binding a cellular receptor which the S1 protein of the T3D Reovirus used in step (c) cannot bind. In one embodiment, the modification to the S1 protein may comprise, relative to the S1 protein of the Reovirus used in step (c), the addition, deletion, substitution or inversion of one or more amino acids to, or from, the primary S1 amino acid sequence. Advantageously, the modification may comprise a modification to the carboxy terminus of the S1 protein. More preferably, the modification comprises the addition of amino acids to the S1 primary sequence and in one embodiment, the modification comprises the addition of one or more histidine residues to the carboxy terminus of the S1 capsid protein.

In a further embodiment, a Reovirus subjected to the methods of modifying cellular tropism described above, may be used in the study and/or treatment of certain diseases and/or conditions. Among the diseases and/or conditions that it may be possible to study and/or treat are cell proliferation and/or differentiation disorders such as cancer. Since it is known that Reoviruses induce apoptosis in cancerous cells, a Reovirus modified to exhibit a tropism for a particular cell type, may be used to treat cancer.

Advantageously, and in a yet further embodiment, a Reovirus may be further modified to comprise one or more nucleic acid sequence(s) which encode a compound or compounds which may induce cell death or apoptosis or which may inhibit or suppress one or more cellular processes. For example, the compounds or compounds may affect those processes involved in protein production and/or the cell (division) cycle. For example, the Reovirus genome may be further modified to include nucleic acid sequences which encode compounds—such as, for example, antisense oligonucleotide sequences, siRNA and/or iRNA sequences which interfere or inhibit normal cellular processes. In a further embodiment, the modified genome may comprise nucleic acid sequences which encode compounds which have a cytotoxic, apoptotic and/or inhibitory effect upon a cell. In this way, Reovirus particles modified in accordance with the present invention may be used to treat certain diseases or conditions.

In a further embodiment, the Reovirus genome may be modified so as to comprise nucleic acid sequences which encode one or more compound(s) which permit detection within a cell. For example, the modified genome may comprise nucleic acids which encode fluorescent compounds, such as GFP or the like.

In a yet further embodiment, the present invention provides a method for modifying the Sigma-1 (S1) capsid protein of Reovirus type 3, strain Dearing (T3D), said method comprising the steps of:

(a) introducing a lentiviral vector comprising a cDNA encoding a modified T3D S1 protein into a cell;

(b) infecting the cell with a T3D Reovirus; and

(c) maintaining the cell under conditions which induce the production of modified T3D virus having a modified S1 protein;

wherein said modified T3D virus having a modified S1 capsid protein further comprises, relative to the T3D Reovirus used in step (b), a modified genome encoding the modified S1 capsid protein.

Typically, the T3D Reovirus used in step (b) is a wild-type, mutant, variant or modified form of the T3D subjected to the above described method.

In a fourth aspect, there is provided a modified virus belonging to the Reoviridae family produced by the methods described herein.

In a fifth aspect, there is provided a modified Reovirus type 3, strain Dearing (T3D), said virus comprising a modified S1 capsid protein comprising at least one histidine residue at the carboxy terminus thereof.

In a sixth aspect, there is provided a method of making a Reovirus type 3, strain Dearing (T3D) comprising a modified S1 capsid protein, said method comprising the steps of:

(a) introducing a cDNA encoding a modified T3D S1 capsid protein into a cell;

(b) infecting the cell with a T3D Reovirus; and

(c) maintaining the cell under conditions which induce the production of modified T3D virus;

wherein said modified T3D virus comprises, relative to the wild-type virus, the modified S1 capsid protein.

Typically, the T3D Reovirus used in step (b) is a wild-type or a mutant, variant or modified form of the T3D subjected to the above described method.

In a seventh aspect, the present invention provides methods of propagating Reoviruses. These methods may require the modification of one or more of the components of a Reovirus in accordance with any of the methods described herein and the subsequent contacting of the modified Reovirus with a cell (for example a modified cell) which expresses a moiety (such as a proteinaceous compound, for example, an antibody or the like) capable of binding or interacting with the modified component of the modified Reovirus. Advantageously, a Reovirus may be modified so as to comprise a modified capsid component capable of interacting with or binding to a compound or moiety expressed by or present on a cell. In this way, via an interaction with (or binding between) the moiety of a cell and the modified component of the Reovirus, the cell may be infected by the modified Reovirus. One of skill in the art will appreciate that by maintaining the modified cell under conditions which permit the production/generation of new virus, it may be possible to propagate the Reovirus.

As such, the present invention provides a method of propagating a modified Reovirus, said method comprising the steps of

(a) contacting a Reovirus modified in accordance with any of the methods described herein with a cell comprising a moiety capable of binding to or interacting with the modified Reovirus under conditions which permit infection of the cell by the modified Reovirus; and

(b) maintaining the cell under conditions which induce the production of modified Reovirus.

In view of the above, and in one embodiment, there is provided a method of propagating a modified Reovirus, said method comprising the steps of:

(a) modifying the S1 capsid protein in accordance with any of the methods described herein such that it comprises at least one histidine residue at the carboxy terminus thereof

(b) contacting the modified Reovirus with a cell modified to express a moiety capable of binding the at least one histidine residue; and (c) maintaining the cell under conditions which induce the production of modified virus.

Preferably the modified Reovirus is a modified Reovirus T3D and the “cell” is derived from a glioblastoma cell line. In one embodiment the cell is a U118MG cell.

Advantageously the moiety capable of binding the at least one histidine residue is a peptide, such as, for example, an antibody. The term “binding moiety” may also be taken to encompass, histidine binding fragments/portions of any such peptides or antibodies. For example, and in the case of an antibody, the fragment may comprise one or more of the heavy and/or light chains and/or a F(ab) and/or F(ab)₂ fragment. For example, the binding moiety may be a single chain antibody.

One of skill in the art will appreciate that, despite the lack of a native Reovirus receptor (for example the JAM-A receptor), modified reovirus carrying the HIS-modified S1 capsid protein can infect and be propagated in cells (such as U118MG cells) which have been modified to express a single chain antibody that interacts with the at least one histidine residue of the modified S1 capsid protein.

In one particular embodiment, the method according to the seventh aspect may permit the propagation of Reovirus which, in addition to the modification of a capsid protein, further comprises a modification to one or more other capsid proteins. For example, the Reovirus to be propagated may comprise a modification adding at least one histidine residue to the carboxy terminus of the S1 capsid protein as well as one or more additional modifications to the same or an alternate capsid protein. Such additional modifications may include, for example deletions, insertions and or replacement, to or of one or more of the amino acids comprising the capsid (for example S1) protein(s) responsible for interacting with a native Reovirus receptor. It is to be understood that a “native” Reovirus receptor may be regarded as the receptor normally bound by the Reovirus in order to infect a cell. Such a receptor may be present on normal, healthy cells. In the case of Reovirus T3D, the native receptor may be regarded as JAM-A. By modifying the amino acid sequences responsible for interacting with a native Reovirus receptor, it may be possible to prevent or inhibit binding, interaction and/or an association between the modified Reovirus and the native Reovirus receptor.

Accordingly, and in a further embodiment, the method according to the seventh aspect may relate to a method of propagating a modified Reovirus comprising a modification which adds at least one histidine residue to the carboxy terminus thereof and a further modification to a capsid protein which alters the amino acids which interact with a native Reovirus receptor.

Advantageously, the further modification may comprise a modification to amino acids Asn369 to Glu384 of the S1 protein of Reovirus T3D. One of skill will appreciate that these particular residues have been suggested to interact with JAM-A (Campbell, et al. et al., (2005) Junctional Adhesion Molecule A Serves as a Receptor for Prototype and Field-Isolate Strains of Mammalian Reovirus. (JOURNAL OF VIROLOGY, 79: 7967-7978).

The above-described method may be used to propagate a virus which, in addition to carrying a histidine modification to the carboxy terminus of the S1 protein, also comprises a modification which introduces into a capsid protein a modification which prevents the virus interacting, binding or otherwise associating with a native receptor. Such viruses may be useful in the treatment of diseases such as cancer as they may specifically target tumour cells as opposed to healthy cells.

In an eighth aspect, there is provided a method of isolating modified Reovirus particles, said method comprising the step of contacting a modified Reovirus having at least one modified capsid component with a moiety capable of binding to or interacting with the at least one modified capsid component under conditions which permit binding between the at least one modified capsid component and the moiety capable of binding to or interacting with the at least one modified capsid component. For example, the method may comprise the step of contacting a modified Reovirus having one histidine residue at the carboxy terminus of the S1 protein with a histidine binding moiety under conditions which permit binding between the at least one histidine residue and the histidine binding moiety.

One of skill in the art will understand that the histidine binding moiety may be any one of the moieties described above. Additionally or alternatively, the histidine binding moiety may comprise a metal ion, such as a nickel ion. Preferably the metal ion may be bound or otherwise immobilised to some form of support substrate such as, for example sepharose, glass, plastic, nitrocellulose, agarose or the like.

The histidine binding moiety may be provided in the form of a column. By way of example, the column may comprise sepharose coupled or conjugated to, or with, a nickel ion.

The method may comprise a wash step during which any modified Reovirus not bound to the histidine binding moiety is removed.

In this way, modified Reovirus may be isolated and/or concentrated from an aqueous solution, cell lysate or the like.

In a ninth aspect, the present invention provides a use of a modified Reovirus produced by any of the methods described herein in the preparation of a vaccine against diseases caused or contributed to by members of the Reoviridae.

In a tenth aspect, there is provided a use of a modified Reovirus produced by any of the methods described herein in the manufacture of a medicament for the treatment of cell proliferation and differentiation disorders such as, for example, cancer.

### DETAILED DESCRIPTION

The present invention will now be described by reference to the following Figures which show:

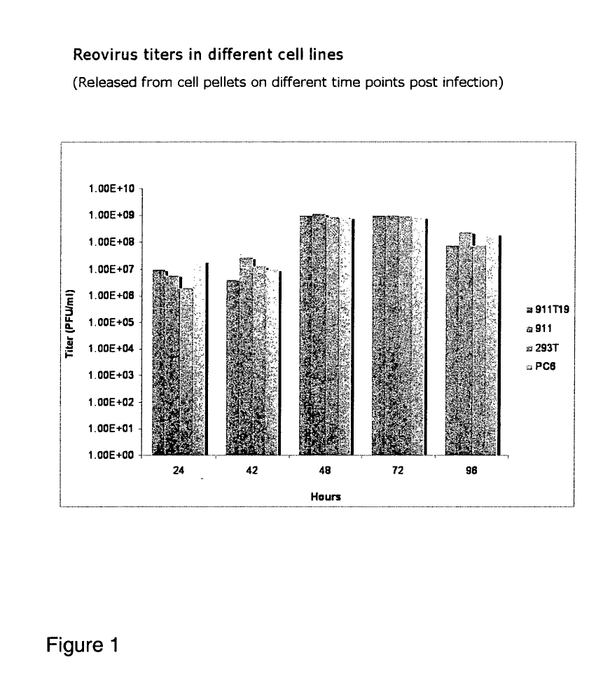

FIG. 1: Reovirus yields in different cell lines

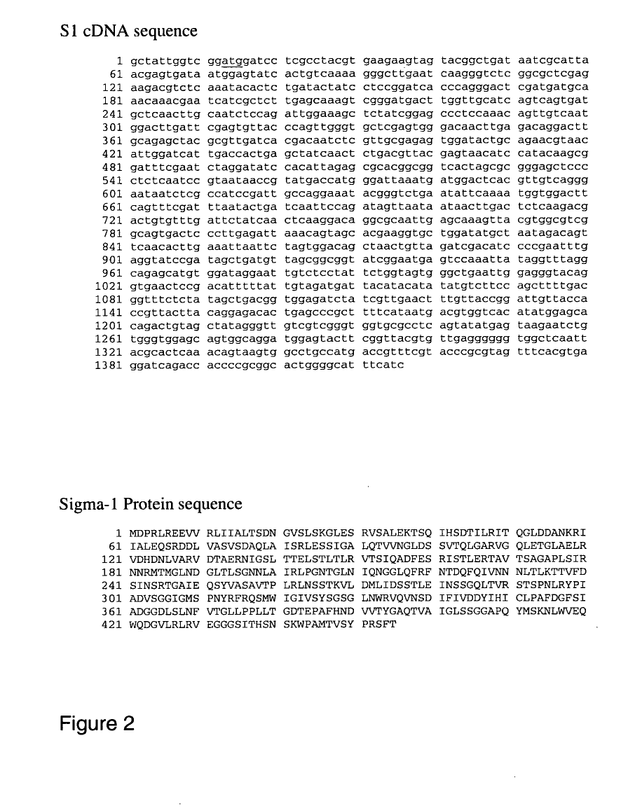

FIG. 2: S1 cDNA Sequence and the amino acid sequences of the Sigma1 protein encoded by it.

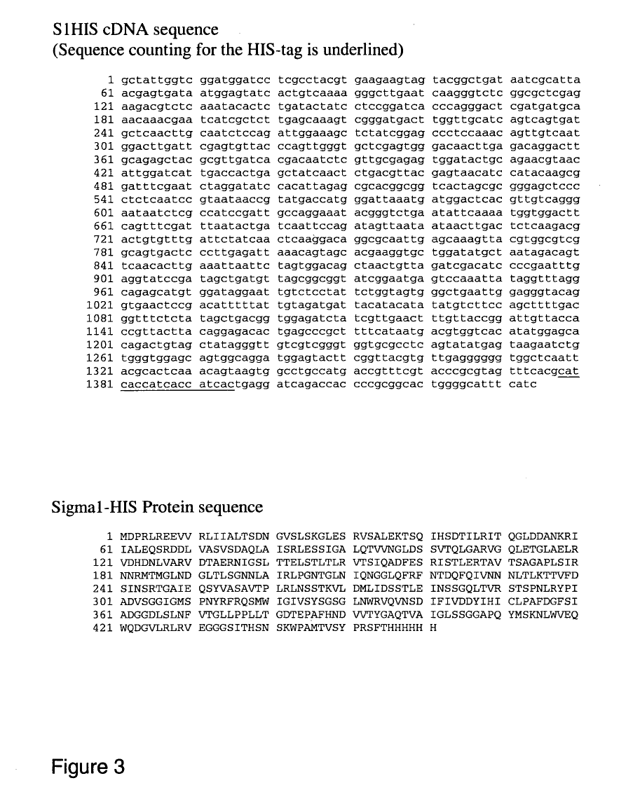

FIG. 3: S1HIS cDNA Sequence and the amino acid sequences of the sigma 1-HIS protein encoded by it.

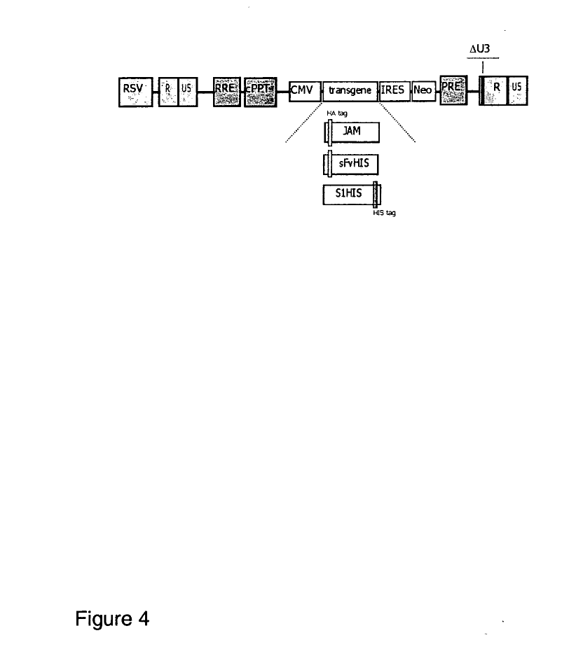

FIG. 4: Schematic representation of the lentivirus constructs encoding HAJam-A, scFvHIS and S1HIS)

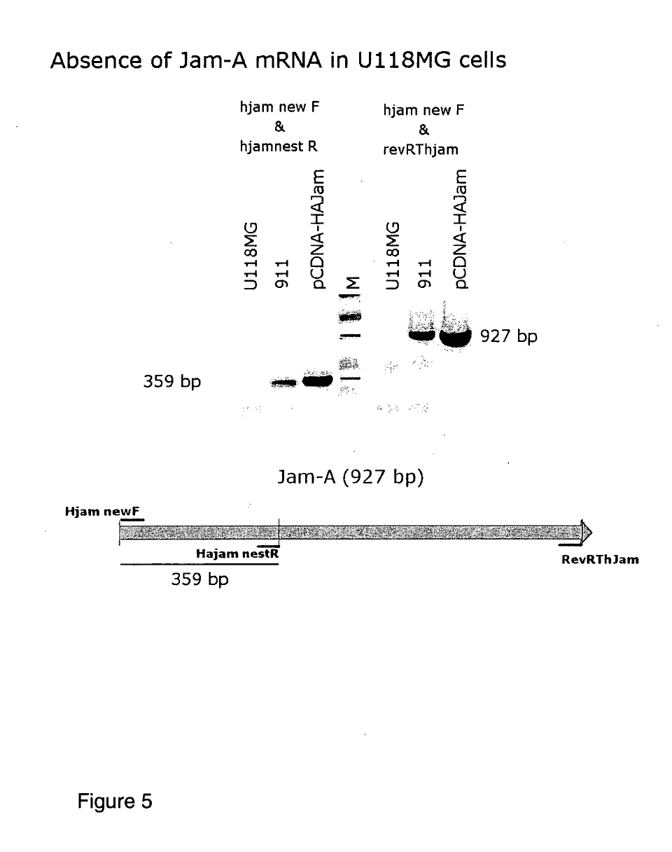

FIG. 5: Reverse-transcriptase PCR analysis demonstrating the absence of Jam-A mRNA in U118MG cells. The lower part illustrate the location of the primes relative to the Jam-A mRNA

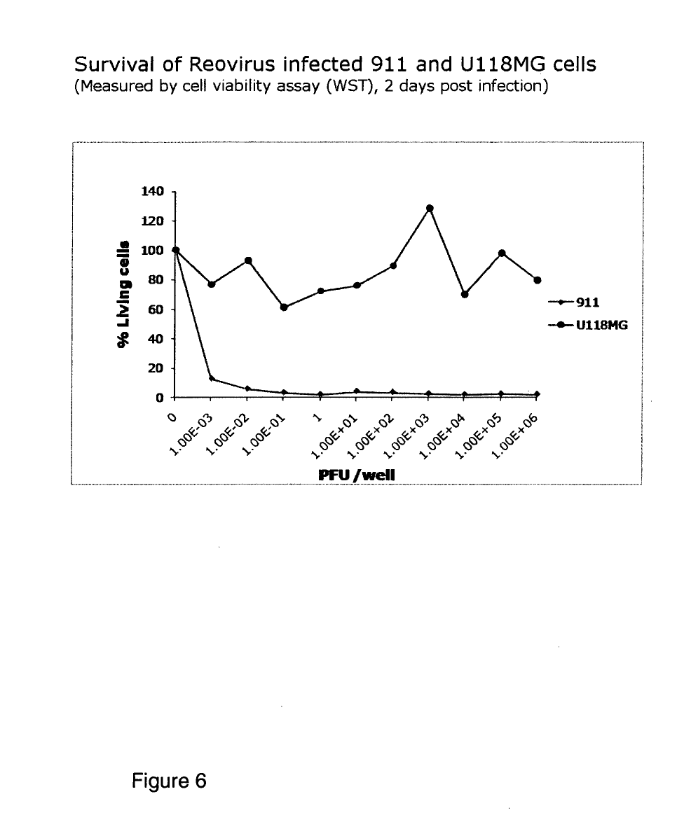

FIG. 6: Survival of Reovirus T3D infected 911 and U118MG cells as determined with a WST cell viability assay.

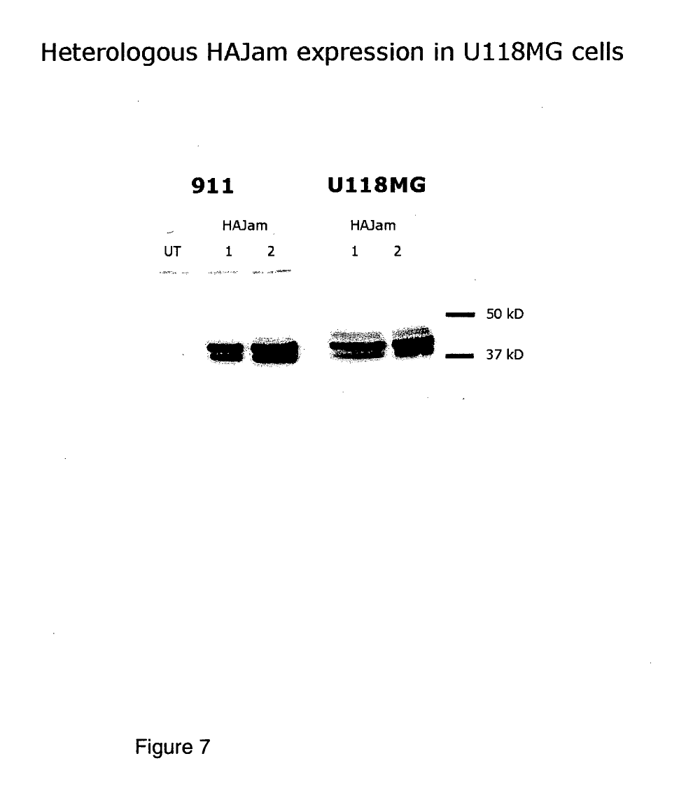

FIG. 7: Heterologous expression of HAJam in U118MG cells as detected by Western analysis with a HA antiserum.

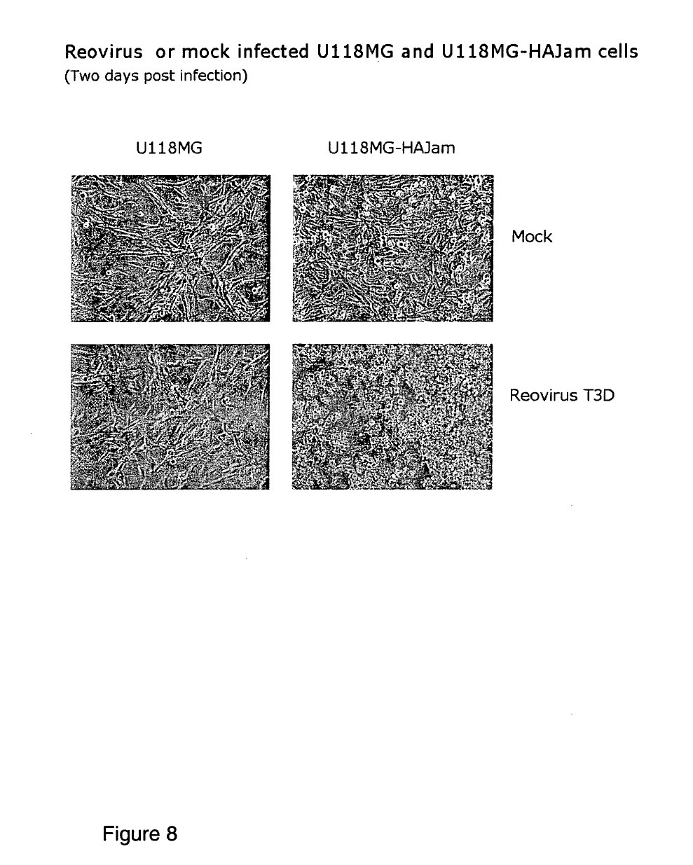

FIG. 8: Cyopathic effects in U118MG-HAJam cells and U118MG cells two days post Reovirus T3D infection.

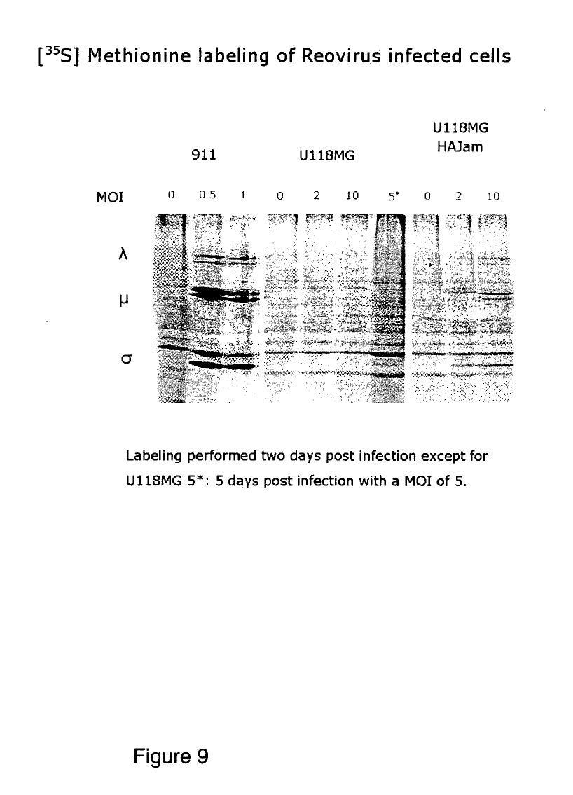

FIG. 9: [35S]-methionine labeling of reovirus T3D infected cells detects the Lambda, Sigma and Mu classes of reovirus proteins as indicated.

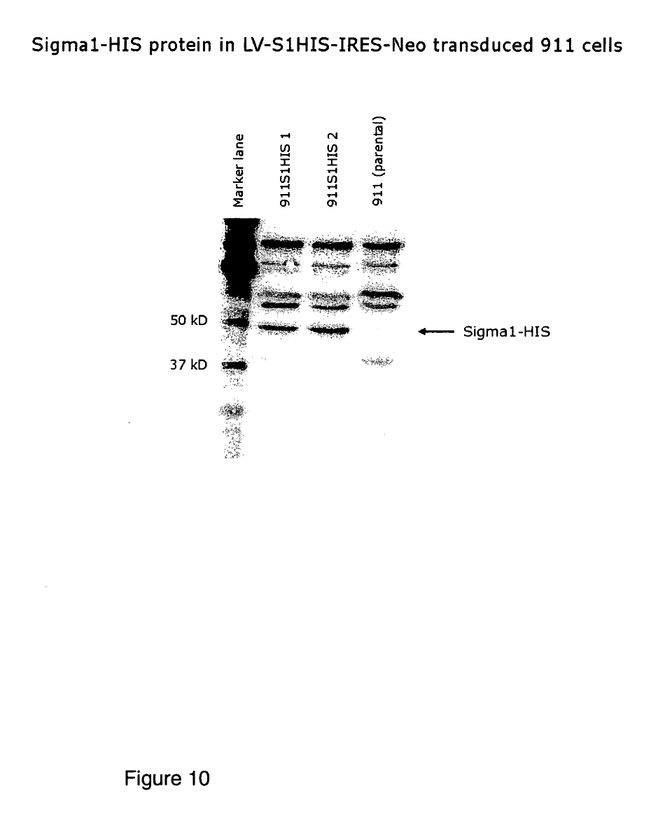

FIG. 10: Sigma1-HIS protein in 911 cells transduced with LV-S1HIS-IRES-Neo. As detected by western analysis with an anti-HIS antiserum

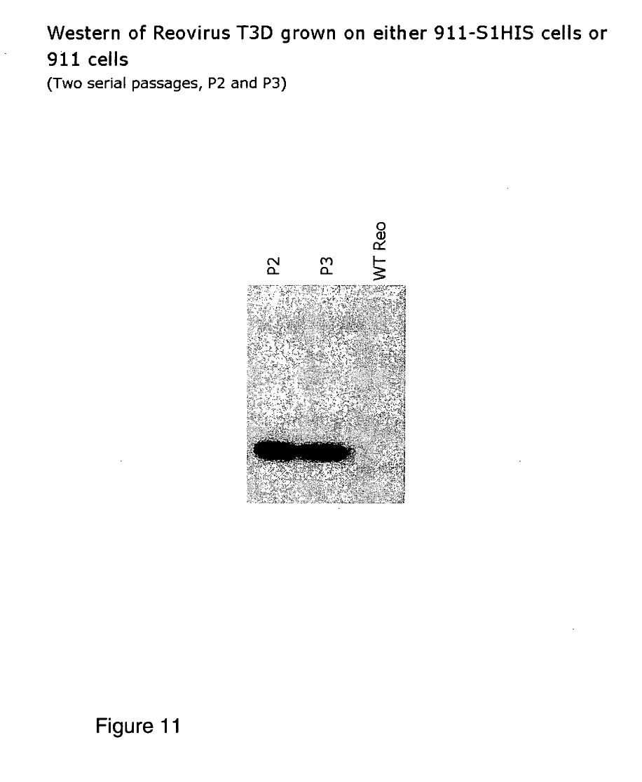

FIG. 11: Western analysis on protein extracts from Reovirus T3D passaged 2(P2) or three (P3) times on 911-S1HIS cells or as control on 911 cells. The western analysis was performed with the penta-HIS serum to detect the presence of the HIS-tag containing Sigma 1 protein.

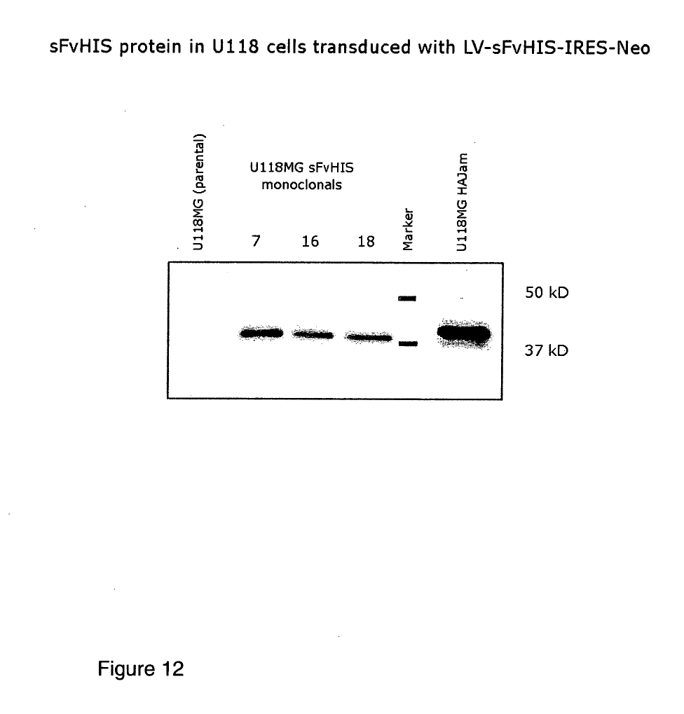

FIG. 12: Western analysis on protein extracts of U118MG cells infected with LV-scFvHIS-IRES-Neo cells. The western analysis was performed with the anti HA serum to detect the presence of the HA-tagged scFvHIS in the transduced cells.

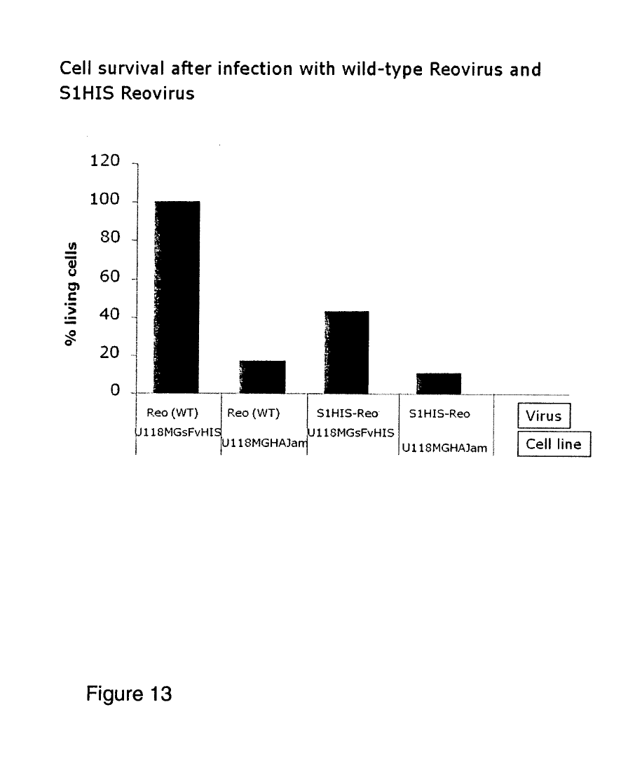

FIG. 13: Cell survival after infection with wild-type Reovirus T3D and the sigma1-HIS-loaded reoviruses, as detected with the WST cell-survival assay.

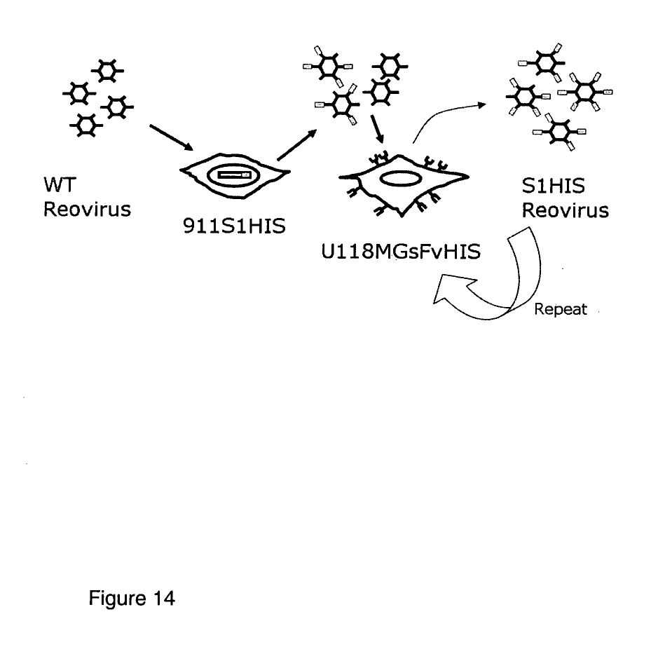

FIG. 14: Schematic outline of the selection system to enrich the Reovirus T3D that acquired the S1-HIS genome segment.

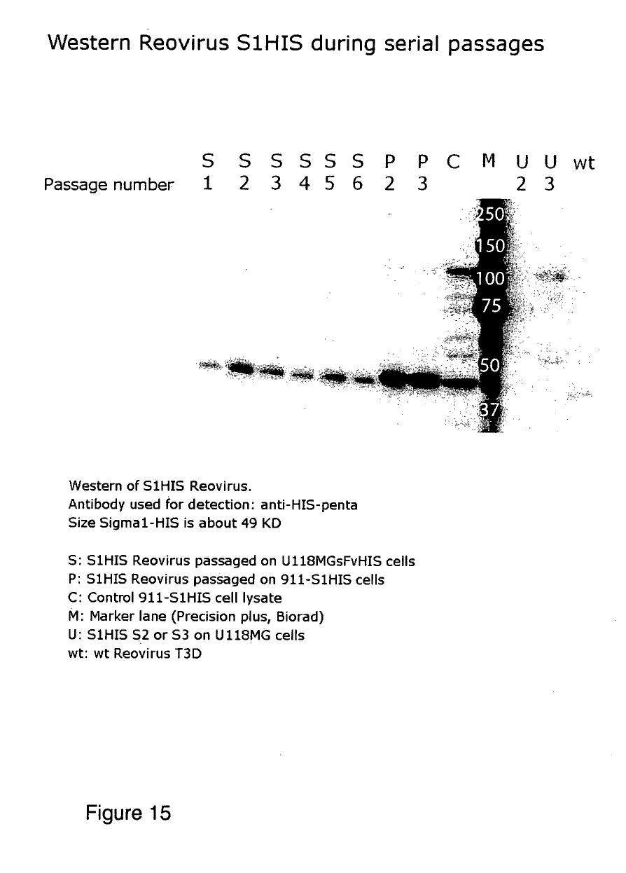

FIG. 15: Western analysis of reovirus T3D during serial passaging (P) and selection (S) on 911-S1His cells and U118MG-scFvHIS cells, respectively, using the pentaHIS serum to detect the HIS-tagged sigma 1 proteins. M=molecular weight marker, wt a sample of wild type reovirus T3D isolated from 911 cells.

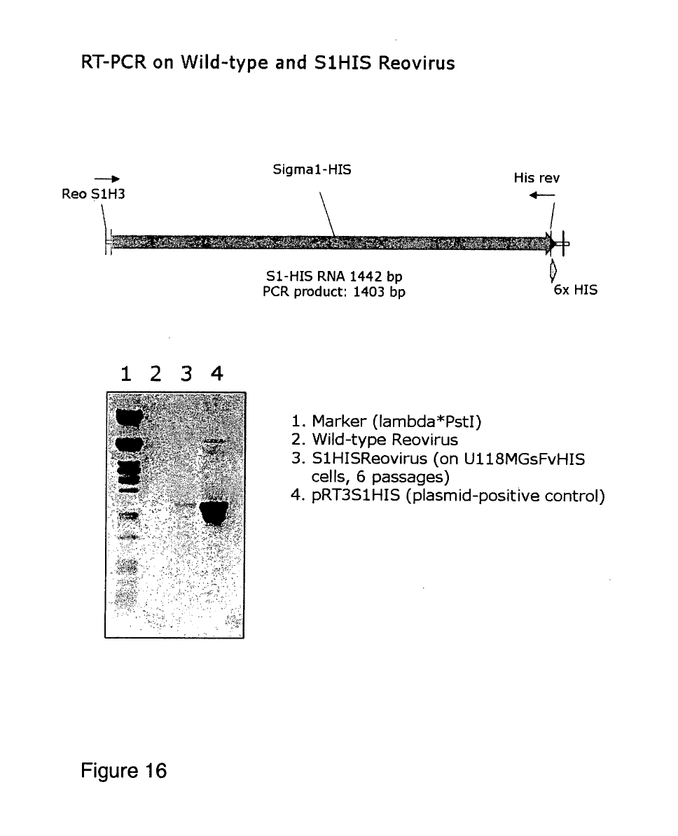

FIG. 16: Reverse-transcriptase PCR to detect the modified S1 genome segment on wild-type Reovirus T3D and the S1-HIS reoviruses that had been selected on the U118MG-scFvHIS cells.

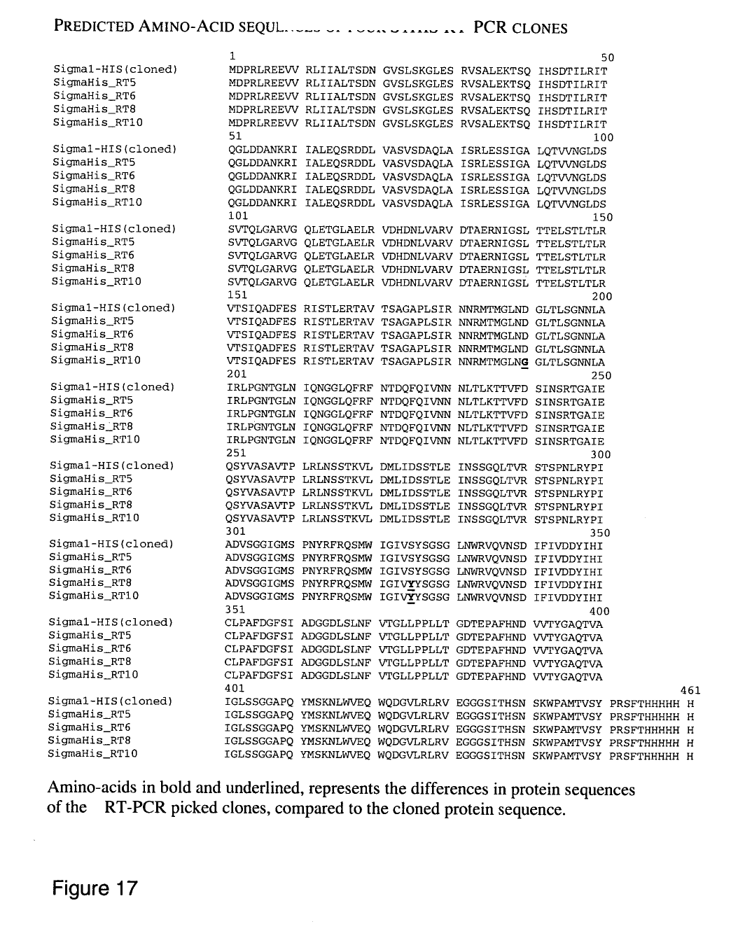

FIG. 17: Amino-acid sequence of the Sigma1_HIS proteins encoded by the S1-HIS segment from reovirus selected for the presence of the HIS-tag on U118MG-scFvHIS cells. The sequences from 4 isolates RT5, RT6, RT8 and RT10 are compared with the Sigma1-HIS that was expressed in the 911 cells (top line).

This disclosure describes the use of the invention for engineering a heterologous stretch of amino acids in the Sigma-1 protein or Reovirus T3D. These amino acids allow the virus carrying the modified Sigma-1 proteins to bind and utilize a new protein receptor on the outside of the tumor cells. The interaction is functional, as is evident from the observation that reovirus T3D carrying Sigma-1 proteins containing the stretch of amino acids, but not the parental wild-type reovirus T3D, is able to infect tumor cells that expresses the cognate protein receptor capable of binding said stretch of amino acids. The reovirus T3D carrying Sigma-1 proteins containing the stretch of amino acids, but not the parental wild-type reovirus T3D could be propagated on the tumor cell line that expresses the cognate protein receptor capable of binding said stretch of amino acids.

The method of our invention relies on expression of a modified Reovirus T3D genome segment using conventional eukaryotic gene expression vectors. In this disclosure the applicants modified the Sigma1 genome segment to encode a Sigma1 protein that carries a carboxy-terminal extension consisting of a tract of 6 histidines. The expression cassette was constructed in such a way that the mRNA starts at the genuine CAP site of the plus-strand Sigma-1 RNA. In contrast to the wild-type S1 genome segment, the modified version was not truncated at the normal 3′ end of the plus-stand RNA but extended and contains a polyA tract. Any conventional RNA polymerase II-dependent transcription cassette can achieve this. In the current form, a standard lentiviral vector was used. With the aid of standard lentiviral vector methods (Carlotti et al., 2004), the expression cassette was transferred into so called 911 cells. In the resulting cells, wild-type reovirus T3D was propagated for 3 passages. The resulting virus stock was used to infect U118MG cells expressing on their surface a single-chain (scFv) antibody which binds HIS-tags. U118MG cells lack the normal reovirus T3D receptor Jam-A. Neither U118MG cells, nor its scFvHIS-receptor-expressing derivatives can be infected by wild-type reovirus. However, the modified reovirus T3D, which contains the HIS-tagged S1 proteins, can use the scFvHIS-receptor as a surrogate receptor and can be propagated in these cells. We confirmed the presence of the HIS-tag in the progeny virus by western blotting, and by nucleotide sequence analyses of the S1 segment. Taken together our data demonstrate (i) the feasibility of reverse genetics of Reoviridae with polyadenylated mRNAs, (ii) that genetic retargeting of Reoviridae is feasible, and (iii) that the C-terminus of the S1 protein is a useful locale for the insertion of host-range modifying mutations. This will be directly useful for generating more effective oncolytic reoviruses, and will facilitate the development of new vaccines for pathogenic Reoviridae.

### Primers

TABLE 1
Primers used in this study

| Primers | Sequences |
|---|---|
| hjam new F | ATGGGGACAAAGGCGCAAGTC |
| revRThjam | CACCAGGAATGACGAGGTC |
| hjamnest R | ATACAAGTGTATGTCCCAGTGTCTT |
| Reo S1N1 | ATTGCGGCCGCGATGAAATGCCCCAGTGCCG |
| Reo S1H3 | CCAAGCTTGCTATTGGTCGGATGGATCCTCG |
| HisReoS1 M2 | GCAGGGTGGTCTGATCCTCAGTGATGGTGAT |
| | GGTGATGCGTGAAACTACGCGGGTA |
| SigmaEnd Rev | GATGAAATGCCCCAGTGCCGCGGGGTGGTCT |
| | GATCCTCA |
| S1endRev | GATGAAATGCCCCAGTGC |
| S1testFor | GAGCATGTGGATAGGAATTG |
| His Rev | GTGATGGTGATGGTGATG |
| M13 For | GTAAAACGACGGCCAG |
| M13 Rev | CAGGAAACAGCTATGAC |

Underlined are restriction sites (NotI and HindIII) and the sequence of the HIS-tag

### EXAMPLE 1

### Construction of Vectors for the Heterologous Expression of Reovirus Receptors and the Reovirus Sigma 1

In the first experiment Reovirus T3D propagated on mouse L-cells. Five days post-infection the progeny virus was released from the infected cells by freeze-thawing the medium and resuspended cells in it. An aliquot of this lysate was used to infect 911 cells (Fallaux et al., 1996), a SV40 Large-T expressing clone of 911 cells, PER.C6 cells (Fallaux et al., 1998), and 293T cells, originally referred to as 293/tsA1609neo (DuBridge et al., 1987). After initial infection (2 hours at 37° C., 5% CO₂), the medium was replaced with normal Dulbecco's modified Eagles medium (DMEM) containing 10% fetal calf serum (FCS), and incubation was continued. The cytopathic effects were apparent in all reovirus T3D infected cell lines 48 hours post infection. At various time points the culture medium was harvested and the cells were resuspended in phosphate-buffered saline (PBS) with 2% FCS at a density of 10⁹/mL. Virus was released from the cells by freeze-thawing for three cycles. The lysates were cleared by centrifugation at 1200×g in a table-top centrifuge for 10 minutes. The concentration of viruses was determined by performing standard plaque assays on 911 cells as was described previously for adenovirus vectors (Fallaux et al., 1996). The data represented in FIG. 1 show that all cell lines tested produced reasonable amounts of reovirus T3D. The highest yields were obtained in 911 cells 48 hrs post-infection. For convenience, cell line 911 was used as standard cell line for virus production and quantization by plaque assay.

To obtain a complementary DNA (cDNA) clone of the Sigma 1 genome segment, 911 cells were infected with ReovirusT3D at 5 plaque-forming units (pfu)/cell in DMEM/2% FCS. After initial infection (2 hours at 37° C., 5% CO₂), the medium was replaced with normal DMEM containing 10% FCS. RNA was extracted from the infected cells 24 hours post infection using the Stratagene Absolutely RNA RT-PCR Miniprep Kit according to the manufacturer's protocol. With primer pair ReoS1/H3 and ReoS1/N1 (table 1) and employing Superscript II Reverse Transcriptase from Invitrogen, the S1 genome segment was copied to complementary DNA (cDNA) and amplified by Polymerase Chain Reaction (PCR) using Taq polymerase, obtained from Promega. After agarose gel electrophoresis, the S1-DNA fragment was purified with the JETsorb gel extraction kit (Genomed), and digested with restriction endonucleases HindIII and NotI. The resulting fragment was cloned in HindIII and NotI-digested plasmid pcDNA3.1+. The resulting ligation mixture was used to transform Escherichia coli strain TOP10F′, and a clone containing a plasmid with the expected structure, designated pcDNART3S1, was isolated and expanded. Plasmid DNA from clone pcDNART3S1 was used for sequence analysis with primer pair ReoS1/H3 and ReoS1/N1 at the Leiden Genome Technology Center. The sequence representing the cDNA of the S1 segment is represented in FIG. 2. The conceptional translation initiation sequence is underlined. The predicted amino acid sequence of the Sigma-1 protein is given.

For retargeting reoviruses to alternative receptors, a new peptide ligand can be included in the viral capsid. One option is to incorporate the codons encoding such ligand in one of the gene segments coding for a capsid component. In the choice of the capsid component, and in the location it is essential to choose a site for inserting the codons for the ligand in such way that in the virus particle the ligand is accessable to the targeted receptor and that no essential structure or function of the modified capsid component is disturbed. Therefore we opted to insert the ligand into the Sigma-1 protein. The crystal structure of part of the Sigma-1 protein of reovirus T3D is known (Chappell et al., 2002b), and deposited in the Brookhaven Protein data bank as 1KKE, DOI 10.2210/pdb1kke/pdb.

The artificial ligand was inserted at the Carboxyl terminus of the Sigma-1 protein, since this region is located in the head domain close to the region that is postulated to interact with the Jam-A protein which serves as a natural receptor for reovirus T3D. (Chappell et al., 2002b) In addition, the carboxyl-terminus of Sigma 1 is positioned in such way that the terminal amino acids are pointing outward. Therefore, it was speculated that fusion of additional of amino acids at the carboxyl terminus should not affect the spatial structure of the head domain. Furthermore, we postulated that the additional amino-acids would be exposed at the surface of the head domain, which would make them assessable and allow them to interact with the targeted receptor.

To add a nucleotide sequence coding for six histidine residues (‘HIS-tag’) to the carboxyl-terminal end of the Sigma-1 protein-coding region a Polymerase Chain Reaction cloning strategy was used. Two different construct were made, both containing the codons for the HIS-tag fused with those for Sigma 1. The first construct contains the HIS-tag but lacks all reovirus sequences downstream of the HIS-tagged Sigma 1. Hence this plasmid lacks the non-coding sequences downstream of the HIS-tagged Sigma 1 protein coding region. The second construct contains the complete cDNA of the segment coding for the HIS-tagged Sigma-1. This constructs contains the entire 3′ untranslated region.

The first plasmid was made by means of Polymerase Chain Reaction, with primer pair His ReoS1 M2 and ReoS1H3 (see table 1 for their sequences). To generate a product containing blunt ends, Pfu polymerase (Promega) was used. The PCR product was digested with HindIII, prior to gel electrophoresis, gel extraction and fragment purification. This product was cloned into plasmid DNA of pcDNA3.1+, which was digested with HindIII and EcoRV. A plasmid with the expected restriction pattern was named pRT3S1HISstop, and used for further studies. The sequence of the fragments inserted in pcDNA3.1+ was determined by DNA sequence analysis. The results confirmed the identity and the expected sequence of the fragment.

Plasmid pRT3S1HISComplete was generated by Polymerase Chain Reaction using pRT3S1HISstop as template and the primer combination of SigmaEndRev and ReoS1H3. The PCR product was digested with HindIII, prior to gel electrophoresis, gel extraction and fragment purification. This product was cloned into plasmid DNA of pcDNA3.1+, which was digested with HindIII and EcoRV. A plasmid with the expected restriction pattern was named pRT3S1HISComplete used for further studies. The sequence of the fragments inserted in pcDNA3.1+ was determined by DNA sequence analysis. The results confirmed the identity and the expected sequence of the fragment. The cDNA sequence of the modified reovirus S1 genome segments is represented in FIG. 3, below the sequence the amino acid sequence of the Sigma-1-HIS protein is represented.

For the generation of cell lines stably expressing heterologous complementary DNA (cDNA) clones, lentiviral vectors can be employed with relative ease. For subsequent experiments four different lentiviral vectors were generated by standard cloning techniques. All lentiviral constructs used in this study were based on the vector made in the pLV-CMV-IRES-NEO vector (Velling a et al., 2006). FIG. 4 gives a schematic representation of the constructs made.

To generate the plasmids pLV-CMV-S1HIS-IRES-NEO and pLV-CMV-S1HISstop-IRES-NEO the constructs pRT3S1HISComplete and pRT3S1HISstop were digested with Eco105I and XbaI and cloned between the Eco105I and XbaI sites in plasmid pLV-CMV-IRES-NEO.

To generate plasmid pLV-CMV-HAJam-IRES-NEO plasmid pcDNA-HAJam (Naik et al., 2001) (kindly provided by Dr. U. P Naik) was digested using restriction endonucleases Eco105I and XbaI and inserted between the Eco105I and XbaI sites in plasmid pLV-CMV-IRES-NEO.

To generate a construct encoding the single-chain HIS-tag receptor, pHISsFv.rec (Douglas et al., 1999) (a kind gift from Dr. D. T. Curiel) was digested with Eco105I and XhoI and inserted between the Eco105I and XhoI sites in plasmid pLV-CMV-IRES-NEO. FIG. 4 gives an overview of the constructs made.

Production of the lentiviral vector stocks was performed exactly as described previously (Carlotti et al., 2004; Velling a et al., 2006) on 293T cells using the calcium phosphate co-precipitation method. All lentiviral vectors were harvested 48 hours after transfection

To generate the cell lines stably expressing the transgenes, suitable dilutions of the different lentiviral vector stocks were added to the cell lines (at a concentrations between 1 and 10 ng p24 per 2500 cells) in the presence of 8 μg/ml polybrene (Sigma Aldrich, Zwijndrecht, The Netherlands) and incubated overnight. The next day the cells were given fresh medium. Forty-eight hours later the cells were detached by trypsinisation and re-plated in medium containing 700 μg/ml G418 (Invitrogen, Breda, The Netherlands) to select for the G418 resistant cell population. Three to five days after the start of the selection, the medium was replaced with medium with 200 μg G418 per ml.

### EXAMPLE 2

### U118MG Cells Resist Reovirus Infection Due to the Absence of its Receptor Jam-A

Several groups have demonstrated that U118MG fully resist reovirus infection. (Wilcox et al., 2001; Yang et al., 2003) To confirm this observation for our U118MG cells, we analyzed the presence of Jam-A mRNA by reverse-transcriptase polymerase chain reaction (rtPCR). As a positive control we include the 911 cells in this analysis.

Primers used for the reverse transcriptase reaction are listed in table 1. U118MG cells and 911 cells were seeded on 5 cm dishes. Upon confluence of the culture, RNA was isolated from the cells using the Absolutely RNA miniprep kit from Stratagene. Six-hundred ng RNA per cell line was used in the first-strand synthesis with SuperScript II (Invitrogen), using the RevRThJam primer (2 pmole per reaction, according to manual). Two μl of the cDNA was used for amplification with the primer combination of RevRThJam and hJam new F to amplify the complete coding region of hJam-A (928 bp). In addition, the primer-pair combination hJamnest R and hjam new F was used for amplifying a shorter product (359 bp). Taq polymerase (Promega) was used for the amplification, with a scheme consisting of the following cycles: 3 min. 95° C., (30 s 95° C.−40 s 58° C.−1 min. 72° C.)×30−10 min. 72° C.−10 min. 4° C.−end. Results are depicted in FIG. 5. Whereas the Jam-A RNA was readily detected in 911 cells, no signal is apparent in the U118MG-derived samples indicating that the MG118 cells lack detectable levels of the Jam-A mRNA.

To confirm that U118MG cells are resistant to reovirus T3D infection, cultures of U118MG cells were exposed to reovirus T3D virus at various multiplicities of infection. As a control in this experiment, cultures of 911 cells were exposed at the same multiplicities. Whereas cytopathic effects were readily observed in the cultures of 911 cells, no changes were apparent upon virus infection in the MG118 cultures, even not at prolonged incubation times. The viability of the cells in these cultures was assayed with the WST cell viability assay (FIG. 6). These data again corroborated that whereas reovirus T3D readily kills 911 cells, U118MG cells fully resist reovirus T3D infection.

To demonstrate that this is due to the absence of the Jam-A receptor, U118MG cells were exposed to the lentiviral vector pLV-CMV-HAJam-IRES-NEO, to force synthesis of the HA-tagged Jam-A protein that serves as the primary receptor for reovirus T3D. Western analysis of protein lysates of the G418-resistant LV-CMV-HAJam-IRES-NEO-transduced cell population, using a HA-specific antiserum demonstrated robust expression of the HA-tagged Jam-A in these cells (FIG. 7). This was further corroborated by immunofluorescence microscopy that revealed that the vast majority of cells in the transduced and selected cell population the HA-Jam-A signal is detectable. Exposure of these cells to reovirus T3D led do rapid development of signs of the cytopathic effects in the HA-Jam expressing U118MG cells, but not in the parental U118MG cells (FIG. 8). This was further corroborated by metabolic labeling of the viral proteins. Infected or mock-infected cell were labeled with Redivue [³⁵S]methionine Pro-mix (200 μCi/ml; Amersham, Roosendaal, the Netherlands) for 4 hours at various time points post infection. Cells were washed once with PBS and lysed in Giordano Lysis Buffer (50 mM Tris-HCl pH 7.4, 250 mM NaCl, 0.1% Triton, 5 mM EDTA) containing a cocktail of protease inhibitors (Complete mini tablets, Roche Diagnostics, Almere, The Netherlands). All the labeling assays were done in 24-wells plates with 5 μl Pro-mix per well, and the volume of lysis buffer was 100 μl per well. Hereof 50 μl was added on a 10% SDS-polyacrylamide gel after addition of Sample Buffer. Gels were dried and exposed to radiographic film, and processed following standard procedures (FIG. 9). The results demonstrated the presence of viral proteins in the 911 cells and the HA-Jam-A expressing U118MG cells, but not in the unmodified U118MG cells. From these data we conclude that U118MG cells resist reovirus T3D infection, and that this is solely due to the absence of the Jam-A protein that can serve as the primary receptor for reovirus T3D infection.

### EXAMPLE 3

### Generation of Sigma 1—Producing 911 Cell Lines

As the next step we generated cell lines producing the reovirus T3D protein. To this end 911 cells were exposed to LV-CMV-S1HIS-IRES-NEO vector viruses at a concentration of 1 to 10 ng p24 per 2500 cells. After selection for the G418-resistant cell population, here named the 911-S1HIS cells, protein lysates were generated from these cells and analyzed by western analysis using the α-Penta-His serum (Qiagen Benelux by, Netherlands) diluted 1:1500, to detect the HIS-tag containing Sigma 1 protein. Results are depicted in FIG. 10. These data demonstrate the presence of a band 49 kDa in the LV-CMV-S1HIS-IRES-NEO transduced cells surviving the G418 selection, but not in the parental 911 cells. From these data we conclude that the 911 cells lines contain significant amounts of the HIS-tagged S1 protein. This implies that S1 over-expression is not toxic to cells.

### EXAMPLE 4

### Functional Incorporation of the Modified Sigma 1 Protein in the Virus Capsid

Subsequently, we infected the 911-S1HIS cells with reovirus T3D at a multiplicity of infection of approximately 10. One day after the appearance of the cytopathic affect, the virus harvested, released the cell bound virus by freeze thawing and used one hundredth of the yield to infect a second culture of 911-S1HIS cells. The virus was passaged on 911-S1HIS three consecutive times. Aliquots of the isolated virus were analyzed by western analysis using the α-Penta-His serum. The results are depicted in FIG. 11. The anti HIS-serum detected a protein migrating at the expected size, suggesting that the reoviruses were able to incorporate the HIS-tagged Sigma 1 protein in their capsid.

To test whether amino-acids inserted in this location could functionally interact with the HIS-tag, U118MG cells were modified to express a single-chain antibody that could interact with the HIS-tag on their cell surface. To this end U118MG cells were exposed to the lentiviral vector LV-CMV-scFvHIS-IRES-NEO. The G418-resistant cell population expressed the single-chain HIS receptor, as was evident from western analysis on protein lysates of the cells using the HA antiserum as a probe (FIG. 12). In the parental U118MG cells the signal is absent. Also, immunofluorescence microscopy revealed a homogenous staining in all cells in the culture demonstrating similar amounts of the protein in all cells. From these data we conclude that the U118MG cells now express the single-chain HIS receptor on their surface.

To test whether the single-chain HIS receptor could be used as a surrogate receptor for the reoviruses T3D carrying the HIS-tagged Sigma-1 protein, the cell line was exposed to increasing amounts of the virus stock, and as control, to equivalent amounts of the parental wild-type reovirus T3D. Whereas U118MG cells expressing the HA-tagged JAM-A are sensitive to both the 911-derived T3D reoviruses and the 911-S1HIS-derived reoviruses, the U118MG-scFvHIS cells are sensitive only to the 911-S1 HIS-derived reoviruses, but not to the 911-derived T3D reoviruses. Signs of the cytopathic effect became overt upon microscopic examination of the U118MG-scFv-HIS cells three days post infection with the T3D virus propagated on the 911-S1HIS line. These data were quantified with the WST cell viability assay (FIG. 13). Taken together these date demonstrate that the scFv-HIS could be used as an artificial receptor in U118MG cells. This allows infection of U118MG cells to proceed independent of the normal reovirus T3D receptor Jam-A. In addition, it confirms the presence of the HIS-tagged Sigma-I protein in the viral capsid, and demonstrates that the HIS-tag is exposed at the viral capsid, allowing infection of the U118MG-scFv-HIS cells.

### EXAMPLE 5

### Incorporation of the Modified S1 Genome Segment in the Reovirus

To test whether reovirus T3D acquired the HIS-tagged S1-genome segment incorporated during propagation on the 911-S1HIS cells, the viruses harvested from the 911-S1HIS cells were used to infect the U118MG-scFvHIS cells. Upon overt signs of the cytopathic effect, the cells were detached from the surface by gently flushing the cells off the dish, and suspended by triturating the cells in the conditioned medium. Viruses were released from the cells by freeze thawing. Subsequently, the reovirus batch was cleared by centrifugation at 2000 rpm in a tabletop centrifuge for 10 minutes. The batch was used again to infect U118MG-scFvHIS cells, and the cells were harvested 4 days post-infection. This procedure was repeated 6 times. The selection scheme is outlined in FIG. 14. Upon serial propagation, signs of the cytopathic effect became more apparent in the U118MG-scFvHIS initially infected with the Reovirus T3D harvested from the 911-S1HIS cells than in cells infected with Reovirus T3D isolated from 911 cells. This suggested that viruses could be propagated on the U118MG-scFvHIS cells. Western analyses on protein lysates the U118MG-scFvHIS cells infected with reoviruses propagated on 911-S1HIS cells and serial passages thereof, demonstrated the presence of HIS-tagged S1 protein in the lysates (FIG. 15).

To verify the presence of the HIS-tagged S1 genome segment, a reverse transcriptase Polymerase Chain Reaction (rtPCR) analysis was performed on RNA isolated from the serially passaged Reovirus T3D as passage seven. Upon signs of the cytopathic effect in the U118MG-scFvHIS cells infected with the serially viruses, RNA was isolated from the cells using the Absolutely RNA miniprep kit from Stratagene. Six-hundred ng RNA per cell line was used in the first-strand synthesis with SuperScript II (Invitrogen), using the His Rev primer (2 pmole per reaction, according to manual). Two μl of the cDNA was used for amplification with the primer combination of His Rev and ReoS1N1 to amplify the complete coding region of the S1 genome segment. Taq polymerase (Promega) was used for the amplification, with a scheme consisting of the following cycles: 3 min. 95° C., (30 s 95° C.−40 s 58° C.−80 s 72° C.)×30−10 min. 72° C.−10 min. 4° C.−end. Results are depicted in FIG. 16. Whereas the HIS-tagged S1 product was readily detected in the U118MG-scFvHIS, no signal is apparent in the U118MG-scFvHIS cells infected with the unmodified 911 cells-derived reovirus T3D. The PCR product was cloned in the plasmid pCRII-TOPO (Invitrogen) according to the manufacturer's instructions. Clones with the fragment inserted were individually expanded and plasmid DNA isolated from these clones was used for DNA sequence analysis with the M13 reverse and M123 forward primers, respectively. Sequence analysis of the cloned PCR product confirmed the presence of the codons for the HIS-tag at the expected position at the C-terminus of the Sigma1-coding. The amino-acid sequences of the sigma-1 proteins encoded by four different S1HIS cDNA clones are represented in FIG. 17. The parental sequence (from FIG. 3) is represented in the top line and designated by Sigma1-His (cloned). The amino acid sequences of clones RT5 and RT6 are identical to the parental clone, but RT8 and RT10 have one and two amino-acids differences, respectively. Nevertheless, the HIS-tag is linked to the carboxyl terminus of sigma 1 in all cases, as expected.

From these data we conclude that upon propagation on the 911 S1HIS cells the reovirus T3D acquired the codons for the HIS tag. This is most likely the result of a reassorting process between the parental wild-type parental S1 genome segment, and the heterologous S1 RNA. However, other mechanisms such as recombination, or template switching during replication, can not be excluded on the basis of the data obtained so far.

The serially propagated virus is incapable of infecting unmodified U118MG cells, demonstrating that the transduction is strictly dependent on the scFv-HIS protein on the cells, which acts as a surrogate receptor.

Taken together our data show generation of retargeted reoviruses can be generated with relative ease by propagation reoviruses on cells that contain polyadenylated mRNAs that are embed a reovirus S1 genome segment. The mRNA expressed in the cells is single-stranded, and contains the entire plus-strand RNA of the S1 genome segment. However, whereas at the 5′ end the heterologous S1 mRNA initiates at or near the position of the bona-fide S1 genome segment, the 3′ end is significantly extended and contains the IRES sequence, the NEO gene, the hepatitis B virus (HBV) derived post-transcriptional regulatory element (PRE), and part of the Human Immunodeficiency Virus type 1 (HIV-1) Long Terminal Repeat. It is evident that the presence of the 3′ extension on the plus strand of the S1-genome segment does not interfere with acquisition of the retargeting mutation.

### REFERENCE LIST

Anonymous, 2007. http://www.oncolyticsbiotech.com/.
Carlotti, F., Bazuine, M., Kekarainen, T., Seppen, J., Pognonec, P., Maassen, J. A., Hoeben, R. C., 2004. Lentiviral vectors efficiently transduce quiescent mature 3T3-L1 adipocytes. Mol. Ther. 9, 209-217.
Chappell, J. D., Gunn, V. L., Wetzel, J. D., Baer, G. S., Dermody, T. S., 1997. Mutations in type 3 reovirus that determine binding to sialic acid are contained in the fibrous tail domain of viral attachment protein sigma1. J. Virol. 71, 1834-1841.
Chappell, J. D., Prota, A. E., Dermody, T. S., Stehle, T., 2002a. Crystal structure of reovirus attachment protein sigma1 reveals evolutionary relationship to adenovirus fiber. EMBO J. 21, 1-11.
Chappell, J. D., Prota, A. E., Dermody, T. S., Stehle, T., 2002b. Crystal structure of reovirus attachment protein sigma1 reveals evolutionary relationship to adenovirus fiber. EMBO J. 21, 1-11.
Coffey, M. C., Strong, J. E., Forsyth, P. A., Lee, P. W., 1998. Reovirus therapy of tumors with activated Ras pathway. Science 282, 1332-1334.
Douglas, J. T., Miller, C. R., Kim, M., Dmitriev, I., Mikheeva, G., Krasnykh, V., Curiel, D. T., 1999. A system for the propagation of adenoviral vectors with genetically modified receptor specificities. Nat. Biotechnol. 17, 470-475.
DuBridge, R. B., Tang, P., Hsia, H. C., Leong, P. M., Miller, J. H., Calos, M. P., 1987. Analysis of mutation in human cells by using an Epstein-Barr virus shuttle system. Mol. Cell Biol. 7, 379-387.
Duncan, M. R., Stanish, S. M., Cox, D. C., 1978. Differential sensitivity of normal and transformed human cells to reovirus infection. J. Virol. 28, 444-449.
Fallaux, F. J., Bout, A., van, d. V., I, van den Wollenberg, D. J., Hehir, K. M., Keegan, J., Auger, C., Cramer, S. J., van Ormondt, H., van der Eb, A. J., Valerio, D., Hoeben, R. C., 1998. New helper cells and matched early region 1-deleted adenovirus vectors prevent generation of replication-competent adenoviruses. Hum. Gene Ther. 9, 1909-1917.
Fallaux, F. J., Kranenburg, O., Cramer, S. J., Houweling, A., van Ormondt, H., Hoeben, R. C., van der Eb, A. J., 1996. Characterization of 911: a new helper cell line for the titration and propagation of early region 1-deleted adenoviral vectors. Hum. Gene Ther. 7, 215-222.
Hashiro, G., Loh, P. C., Yau, J. T., 1977. The preferential cytotoxicity of reovirus for certain transformed cell lines. Arch. Virol. 54, 307-315.
Hirasawa, K., Nishikawa, S. G., Norman, K. L., Coffey, M. C., Thompson, B. G., Yoon, C. S., Waisman, D. M., Lee, P. W., 2003. Systemic reovirus therapy of metastatic cancer in immune-competent mice. Cancer Res. 63, 348-353.
Kapikian, A. Z., Hoshino, Y., Chanock, R. M., 2001. Rotaviruses. In: D. M. Knipe, P. M. Howley (Eds.), Fields Virology. Lippincott Williams and Wilkins, Philadelphia, pp. 1787-1833.
Komoto, S., Sasaki, J., Taniguchi, K., 2006. Reverse genetics system for introduction of site-specific mutations into the double-stranded RNA genome of infectious rotavirus. Proc. Natl. Acad. Sci. U.S.A. 103, 4646-4651.
Maginnis, M. S., Forrest, J. C., Kopecky-Bromberg, S. A., Dickeson, S. K., Santoro, S. A., Zutter, M. M., Nemerow, G. R., Bergelson, J. M., Dermody, T. S., 2006. Beta1 integrin mediates internalization of mammalian reovirus. J. Virol. 80, 2760-2770.
Naik, U. P., Naik, M. U., Eckfeld, K., Martin-DeLeon, P., Spychala, J., 2001. Characterization and chromosomal localization of JAM-1, a platelet receptor for a stimulatory monoclonal antibody. J. Cell Sci. 114, 539-547.
Nibert, M., Schiff, L. A., 2001. Reoviruses and their replication. In: D. M. Knipe, P. M. Howley (Eds.), Fields Virology. Lippincott, Williams and Wilkins; Philadelphia, pp. 1679-1728.
Norman, K. L., Lee, P. W., 2000. Reovirus as a novel oncolytic agent. J. Clin. Invest 105, 1035-1038.
Roner, M. R., Joklik, W. K., 2001. Reovirus reverse genetics: Incorporation of the CAT gene into the reovirus genome. Proc. Natl. Acad. Sci. U.S.A. 98, 8036-8041.
Roner, M. R., Sutphin, L. A., Joklik, W. K., 1990. Reovirus RNA is infectious. Virology 179, 845-852.
Roy, P., 2001. Orbiviruses. In: D. M. Knipe, P. M. Howley (Eds.), Fields Virology. Lippincott Williams and Wilkins, Philadelphia, pp. 1835-1869.
Shmulevitz, M., Marcato, P., Lee, P. W., 2005. Unshackling the links between reovirus oncolysis, Ras signaling, translational control and cancer. Oncogene 24, 7720-7728.
Smakman, N., 2005. Towards KRAS-dependent therapy. Utrecht.
Smakman, N., van den Wollenberg, D. J., Borel, R., I, Hoeben, R. C., Kranenburg, O., 2005. Sensitization to apoptosis underlies KrasD12-dependent oncolysis of murine C26 colorectal carcinoma cells by reovirus T3D. J. Virol. 79, 14981-14985.
Smakman, N., van den Wollenberg, D. J., Elias, S. G., Sasazuki, T., Shirasawa, S., Hoeben, R. C., Borel, R., I, Kranenburg, O., 2006a. KRAS (D13) Promotes apoptosis of human colorectal tumor cells by ReovirusT3D and oxaliplatin but not by tumor necrosis factor-related apoptosis-inducing ligand. Cancer Res. 66, 5403-5408.
Smakman, N., van der Bilt, J. D., van den Wollenberg, D. J., Hoeben, R. C., Borel, R., I, Kranenburg, O., 2006b. Immunosuppression promotes reovirus therapy of colorectal liver metastases. Cancer Gene Ther. 13, 815-818.
Strong, J. E., Coffey, M. C., Tang, D., Sabinin, P., Lee, P. W., 1998. The molecular basis of viral oncolysis: usurpation of the Ras signaling pathway by reovirus. EMBO J. 17, 3351-3362.
Strong, J. E., Lee, P. W., 1996. The v-erbB oncogene confers enhanced cellular susceptibility to reovirus infection. J. Virol. 70, 612-616.
Strong, J. E., Tang, D., Lee, P. W., 1993. Evidence that the epidermal growth factor receptor on host cells confers reovirus infection efficiency. Virology 197, 405-411.
Tang, D., Strong, J. E., Lee, P. W., 1993. Recognition of the epidermal growth factor receptor by reovirus. Virology 197, 412-414.
Tyler, K. L., 2001. Mammalian Reoviruses. In: D. M. Knipe, P. M. Howley (Eds.), Fields Virology. Lippincott, Williams and Wilkins; Philadelphia, pp. 1729-1746.
Velling a, J., Uil, T. G., de Vrij, J., Rabelink, M. J., Lindholm, L., Hoeben, R. C., 2006. A system for efficient generation of adenovirus protein IX-producing helper cell lines. J. Gene Med. 8, 147-154.
Wilcox, M. E., Yang, W., Senger, D., Rewcastle, N. B., Morris, D. G., Brasher, P. M., Shi, Z. Q., Johnston, R. N., Nishikawa, S., Lee, P. W., Forsyth, P. A., 2001. Reovirus as an oncolytic agent against experimental human malignant gliomas. J. Natl. Cancer Inst. 93, 903-912.
Yang, W. Q., Senger, D., Muzik, H., Shi, Z. Q., Johnson, D., Brasher, P. M., Rewcastle, N. B., Hamilton, M., Rutka, J., Wolff, J., Wetmore, C., Curran, T., Lee, P. W., Forsyth, P. A., 2003. Reovirus prolongs survival and reduces the frequency of spinal and leptomeningeal metastases from medulloblastoma. Cancer Res. 63, 3162-3172.
Zeevi, M., Nevins, J. R., Darnell, J. E., Jr., 1982. Newly formed mRNA lacking polyadenylic acid enters the cytoplasm and the polyribosomes but has a shorter half-life in the absence of polyadenylic acid. Mol. Cell Biol. 2, 517-525.

1. A method for modifying the genome of a virus belonging to the Reoviridae, said method comprising the steps of:
(a) introducing a nucleic acid encoding a modified portion of a Reovirus genome into a cell;
(b) infecting the cell with a Reovirus; and
(c) maintaining the cell under conditions which induce the production of modified virus;
wherein said modified virus comprises, relative to the Reovirus used in step (b), a modified genome comprising the modified portion of the Reovirus genome.

2. The method of claim 1, wherein the virus belonging to the Reoviridae is an Orthoreovirus, Orbivirus, Rotavirus or Coltivirus species.

3. The method of claim 1, wherein one or more of the double-stranded RNA genome segments comprising the Reovirus genome is/are modified.

4. The method of claim 1, wherein a portion or portions of one or more of the double-stranded RNA genome segments is/are modified.

5. The method of claim 1, wherein one or more of the viral components encoded by the genome is/are modified.

6. The method of claim 5, wherein the one or more viral components is/are structural and/or non-structural components.

7. The method of claim 1, wherein the Reovirus genome is modified so as to comprises one or more heterologous nucleic acid sequences.

8. The method of claim 7, wherein the heterologous nucleic acid sequences encode a compound or compounds which induce cell death or apoptosis or which may inhibit or suppress one or more cellular processes.

9. The method of claim 1, wherein the nucleic acid to be introduced into the cell is contained within a RNA polymerase II-dependent transcription cassette.

10. The method of claim 9, wherein the RNA polymerase II-dependent transcription cassette is a lentiviral vector.

11. A method of modifying the cellular tropism of a virus belonging to the Reoviridae, said method comprising the steps of:
(a) introducing a nucleic acid encoding a modified component of a Reovirus into a cell;
(b) infecting the cell with a Reovirus; and
(c) maintaining the cell under conditions which induce the production of modified Reovirus of modified cellular tropism;
wherein said modified Reovirus of modified tropism comprises, relative to the Reovirus used in step (b), the modified component the Reovirus.

12. A method for modifying the Sigma-1 (S1) capsid protein of Reovirus type 3, strain Dearing (T3D), said method comprising the steps of:
(a) introducing a lentiviral vector comprising a cDNA encoding a modified T3D S1 protein into a cell;
(b) infecting the cell with T3D virus; and
(c) maintaining the cell under conditions which induce the production of modified T3D virus having a modified S1 protein;
wherein said modified T3D virus having a modified S1 capsid protein further comprises, relative to the T3D virus used in step (b), a modified genome encoding the modified S1 capsid protein.

13. A modified virus belonging to the Reoviridae family produced by the method of claim 1.

14. A modified Reovirus type 3, strain Dearing (T3D), said virus comprising a modified S1 capsid protein comprising at least one histidine residue at the carboxy terminus thereof.

15. A method of propagating a modified Reovirus, said method comprising the steps of
(a) contacting a Reovirus modified in accordance with the method of claim 1, with a cell comprising a moiety capable of binding to or interacting with the modified Reovirus under conditions which permit infection of the cell by the modified Reovirus; and
(b) maintaining the cell under conditions which induce the production of modified Reovirus.

16. The method of claim 15, wherein the modified Reovirus is a T3D Reovirus comprising a S1 capsid protein modified such that it comprises at least one histidine residue at the carboxy terminus thereof and further wherein the moiety of the cell is capable of binding the at least one histidine residue of the modified S1 capsid protein.

17. The method of claims 16, wherein the modified Reovirus further comprises one or more additional modifications to a capsid protein.

18. The method of claim 17, wherein the additional modification comprises a modification to amino acids Asn369 to Glu384 of the S1 protein of Reovirus T3D.

19. A method of treating diseases such as cancer, comprising administering to a subject in need thereof, a Reovirus propagated by the method according to claim 15.

20. A method of isolating modified Reovirus particles, said method comprising the step of contacting a modified Reovirus having at least one histidine residue at the carboxy terminus of the S1 protein with a histidine binding moiety under conditions which permit binding between the at least one histidine residue and the histidine binding moiety.

21. A vaccine for preventing diseases caused or contributed to by members of the Reoviridae, comprising a modified Reovirus produced by the method according to claim 1.

22. A method of treating a cell proliferation or differentiation disorder in a subject, comprising administering to a subject in need thereof, a Reovirus produced by the method according to claim 1.
